(19) United States
(12) Patent Application Publication
(10) Pub. No.: US 2005/0228345 A1
(43) Pub. Date: Oct. 13, 2005

(54) Single-use safety injector structure

(76) Inventors: Chang-Ming Yang, Miaoli (TW); Chun-Mei Chou, Miaoli (TW)

Correspondence Address: HDSL, 4331 STEVENS BATTLE LANE, FAIRFAX, VA, 22033, US

(21) Appl. No.: 10/972,279

(22) Filed: Oct. 25, 2004

(30) Foreign Application Priority Data
Apr. 12, 2004 (TW) 093205527

(51) Int. Cl. A61M 5/00

(52) U.S. Cl. 604/110; 604/198

(57) ABSTRACT

A single-use safety injector structure includes a syringe needle, a protective cover and a syringe. The syringe needle is connected to the syringe via an insertion portion. The outer periphery of the insertion portion is provided with a gliding block. The gliding block includes a base, a guiding rod and an upper stopping portion. The outer periphery of the protective cover is provided with a gliding track. One or both sides in the vicinity of the front end and rear end of the gliding track are provided with salients. The gliding track is provided with one or more non-return stopping blocks behind and in the vicinity of the rear-end salient. The protective cover is sleeved around the outer periphery of the syringe needle. By means of the guiding rod, the injection needle penetrates through the gliding track of the protective cover. By means of the base, the syringe needle is sustained against the inner wall of the protective cover. By means of the upper stopping portion, the syringe needle is sustained against the external wall of the protective cover.

### BACKGROUND OF THE INVENTION

1. Field of the Invention

The invention relates to a single-use safety injector structure, and in particular, to a single-use safety injector structure which is safely and easily operated without worrying about its repeated use.

2. Description of the Prior Art

Conventionally, an injector is consisted of a syringe needle and a syringe cover. The syringe needle is provided with several lateral pieces extended from sidewalls of a syringe body, and an injection needle is provided in front of the syringe body. The syringe cover is in a cylindrical form having an accommodating aperture at its base. However, in the course of the blood collection by using the above-described conventional injector, since the syringe cover should be removed from the syringe at first before injecting, after completion of the blood collection, the medical practitioner should return the syringe cover, and just at this time, he (she) will be subject to be hurt by the injection needle of the syringe needle if carelessly handled, and hence is susceptible to be infected.

Accordingly, the conventional art mentioned above has still many disadvantages and is not designed perfectly such that it needs to be improved immediately.

In view of the various disadvantages associated with the prior art product, the inventor of this application had devoted to improve it and, after studying intensively for many years, accomplished successfully the present single-use safety injector structure.

### SUMMARY OF THE INVENTION

It is an object of the present invention to provide a single-use safety injector structure characterized in that it is easily and safely operated.

It is another object of the present invention to provide a single-use safety injector structure operated without worrying about its repeated use.

The single-use safety injector structure that can accomplishes the above-described objects according to the invention is consisted of:

a syringe needle having an injection needle inserted into an insertion portion thereof, wherein the outer periphery of the insertion portion is provided with a gliding block, and the gliding block includes a base, a guiding rod and an upper stopping portion;

a protective cover provided with a gliding track on the outer periphery thereof, wherein one or both sides in the vicinity of the front end and rear end of the gliding track are provided with salients, and the gliding track is provided with one or more non-return stopping blocks behind and in the vicinity of the rear-end salient; and

a syringe for inserting the syringe needle via the front end thereof,

wherein the syringe needle is connected to the front end of the syringe via the insertion portion, the protective cover is sleeved around the outer periphery of the syringe needle, by means of the guiding rod, the syringe needle penetrates through the gliding track of the protective cover, by means of the base, the syringe needle is sustained against the inner wall of the protective cover, and by means of the upper stopping portion, the syringe needle is sustained against the external wall of the protective cover.

### BRIEF DESCRIPTION OF THE DRAWINGS

The drawings disclose an illustrative embodiment of the present invention which serves to exemplify the various advantages and objects hereof, and are as follows:

FIG. 1 is a three-dimensional exploded top view of the single-use safety injector structure according to the invention;

FIG. 2 is a three-dimensional exploded view of a first embodiment of the single-use safety injector structure;

FIG. 3 is a three-dimensional exploded view of a second embodiment of the single-use safety injector structure;

FIG. 4 is a three-dimensional exploded view of a third embodiment of the single-use safety injector structure;

FIG. 5 is a three-dimensional out-looking view of a fourth embodiment of the single-use safety injector structure;

FIG. 6 is a three-dimensional exploded view of a fifth embodiment of the single-use safety injector structure;

FIG. 7 is a three-dimensional exploded view of a sixth embodiment of the single-use safety injector structure;

FIG. 8 is a three-dimensional out-looking view of a seventh embodiment of the single-use safety injector structure;

FIG. 9 is a three-dimensional out-looking view of an eighth embodiment of the single-use safety injector structure;

FIG. 10 is a three-dimensional exploded view of a ninth embodiment of the single-use safety injector structure;

FIG. 11 is a three-dimensional exploded view of a tenth embodiment of the single-use safety injector structure;

FIG. 12 is a three-dimensional exploded view of an eleventh embodiment of the single-use safety injector structure;

FIG. 13 is a three-dimensional exploded view of a twelfth embodiment of the single-use safety injector structure;

FIG. 14 is a three-dimensional exploded view of a thirteenth embodiment of the single-use safety injector structure;

FIG. 15 is a three-dimensional exploded view of a fourteenth embodiment of the single-use safety injector structure;

FIG. 16 is a three-dimensional exploded view of a fifteenth embodiment of the single-use safety injector structure;

FIG. 17 is a three-dimensional exploded view of a sixteenth embodiment of the single-use safety injector structure;

FIG. 18 is a three-dimensional cross-sectional view of a seventeenth embodiment of the single-use safety injector structure;

FIG. 19 is a three-dimensional exploded view of an eighteenth embodiment of the single-use safety injector structure;

FIG. 20 is a three-dimensional exploded view of a nineteenth embodiment of the single-use safety injector structure;

FIG. 21 is a three-dimensional exploded view of a twentieth embodiment of the single-use safety injector structure;

FIG. 22 is a three-dimensional exploded view of a twenty-first embodiment of the single-use safety injector structure;

FIG. 23 is a three-dimensional exploded view of a twenty-second embodiment of the single-use safety injector structure;

FIG. 24 is a three-dimensional exploded view of a twenty-third embodiment of the single-use safety injector structure;

FIG. 25 is a three-dimensional exploded view of a twenty-fourth embodiment of the single-use safety injector structure;

FIG. 26 is a three-dimensional out-looking view of a twenty-fifth embodiment of the single-use safety injector structure;

FIG. 27 is a three-dimensional exploded view of a twenty-sixth embodiment of the single-use safety injector structure;

FIG. 28 is a three-dimensional out-looking view of a twenty-seventh embodiment of the single-use safety injector structure;

FIG. 29 is a three-dimensional exploded view of a twenty-eighth embodiment of the single-use safety injector structure;

FIG. 30 is a three-dimensional exploded view of a twenty-ninth embodiment of the single-use safety injector structure;

FIG. 31 is a three-dimensional out-looking view of a thirtieth embodiment of the single-use safety injector structure;

FIG. 32 is a three-dimensional out-looking view of a thirty-first embodiment of the single-use safety injector structure;

FIG. 33 is a three-dimensional exploded view of a thirty-second embodiment of the single-use safety injector structure;

FIG. 34 is a three-dimensional exploded view of a thirty-third embodiment of the single-use safety injector structure;

FIG. 35 is a three-dimensional exploded view of a thirty-fourth embodiment of the single-use safety injector structure;

FIG. 36 is a three-dimensional exploded view of a thirty-fifth embodiment of the single-use safety injector structure;

FIG. 37 is a three-dimensional exploded view of a thirty-sixth embodiment of the single-use safety injector structure;

FIG. 38 is a three-dimensional exploded view of a thirty-seventh embodiment of the single-use safety injector structure;

FIG. 39 is a three-dimensional exploded view of a thirty-eighth embodiment of the single-use safety injector structure;

FIG. 40 is a three-dimensional exploded view of a thirty-ninth embodiment of the single-use safety injector structure;

FIG. 41 is a three-dimensional exploded view of a fortieth embodiment of the single-use safety injector structure;

FIG. 42 is a three-dimensional exploded view of a forty-first embodiment of the single-use safety injector structure;

FIG. 43 is a three-dimensional exploded view of a forty-second embodiment of the single-use safety injector structure;

FIG. 44 is a three-dimensional exploded view of a forty-third embodiment of the single-use safety injector structure; and

FIG. 45 is a three-dimensional exploded view of a forty-fourth embodiment of the single-use safety injector structure.

### DETAILED DESCRIPTION OF THE PREFERRED EMBODIMENT

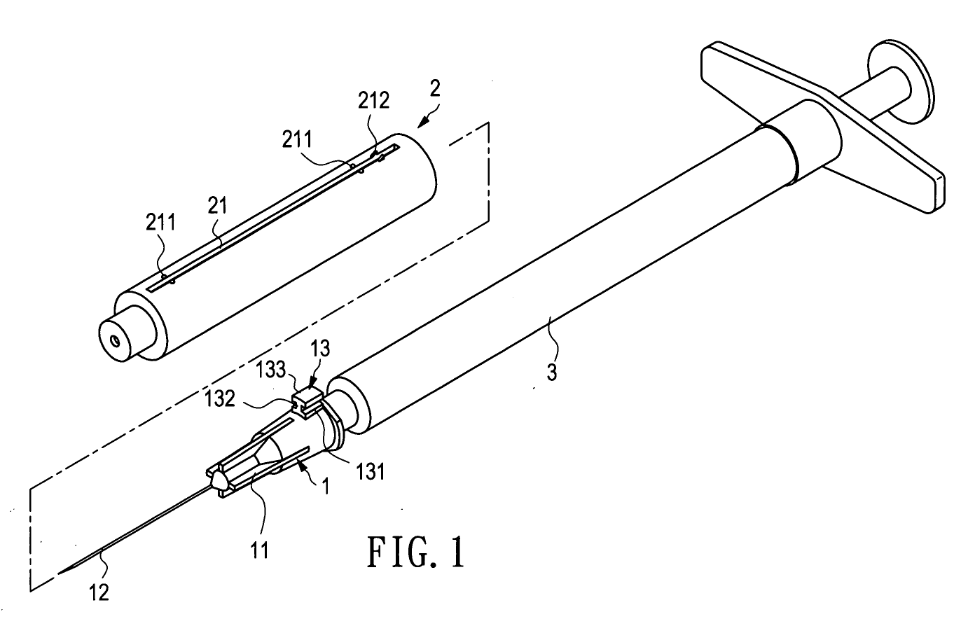

Referring to FIG. 1, the single-use safety injector structure provided by the invention is consisted essentially of:

a syringe needle 1, having an injection needle 12 inserted into an insertion portion 11 thereof, wherein the outer periphery of the insertion portion 11 is provided with a gliding block 13, and the gliding block 13 includes a base 131, a guiding rod 132 and an upper stopping portion 133;

a protective cover 2, provided with a gliding track 21 on the outer periphery thereof, wherein one or both sides in the vicinity of the front end and rear end of the gliding track 21 are provided with salients 211, and the gliding track 21 is provided with one or more non-return stopping blocks 212 behind and in the vicinity of the rear-end salient 211; and

a syringe 3 for inserting the syringe needle via the front end thereof.

The syringe needle 1 is connected to the front end of the syringe 3 via the insertion portion 11. The protective cover 2 is sleeved around the outer periphery of the syringe needle 1. By means of the guiding rod 132, the syringe needle 1 penetrates through the gliding track 21 of the protective cover 2. By means of the base 131, the syringe needle 1 is sustained against the inner wall of the protective cover 2. By means of the upper stopping portion 133, the syringe needle 1 is sustained against the external wall of the protective cover 2. Before the single-use safety injector structure provided by the invention is used, the upper stopping portion 133 of the gliding block 13 is fixed between the rear-end salient 211 on the gliding track 21 of the protective cover 2 and the non-return stopping block 212. When the single-use safety injector structure provided by the invention is used, the only thing to be done is to pull the protective cover 2 backwardly. As such, the upper stopping portion 133 is detached from the rear-end salient 211 of the gliding track 21 and passes through the front-end salient 211 of the gliding track 21, such that the injection needle 12 is exposed from the front end of the protective cover 2. In addition, the rear-end wall of the upper stopping portion 133 of the gliding block 13 fails to move backwardly because it is stopped by the front-end salient 211 of the gliding track 21. Meanwhile, the injection needle 12 can be inserted into the injecting site of the subject so as to perform the injecting or drawing-out action. After the injecting or drawing-out action is completed, the only thing to be done is to push the protective cover 2 forwardly. As such, the upper stopping portion 133 will pass through the front-end salient 211 of the gliding track 21, such that the injection needle 12 is encapsulated within the protective cover 2. The protective cover 2 is continuously pushed forwardly. As such, the upper stopping portion 133 of the gliding block 13 will pass through the non-return stopping blocks 212 at the rear end of the gliding track 21. In addition, the protective cover 2 fails to be pushed backwardly since it is stopped on the front-end wall of the upper stopping portion 133 by means of the non-return stopping blocks 212. Accordingly, the single-use safety injector structure of the present invention can be operated without worrying about its repeated use. Alternatively, the salient 211 and the non-return stopping blocks 212 arranged on the outer periphery of the protective cover 2 can be provided on the inner wall of the gliding track 21 so as to stop the guiding rod 132 of the gliding block 13. Furthermore, the salient 211 and the non-return stopping blocks 212 arranged on the outer periphery of the protective cover 2 can be provided on the inner wall of the gliding track 21 so as to stop the base 131 of the gliding block 13.

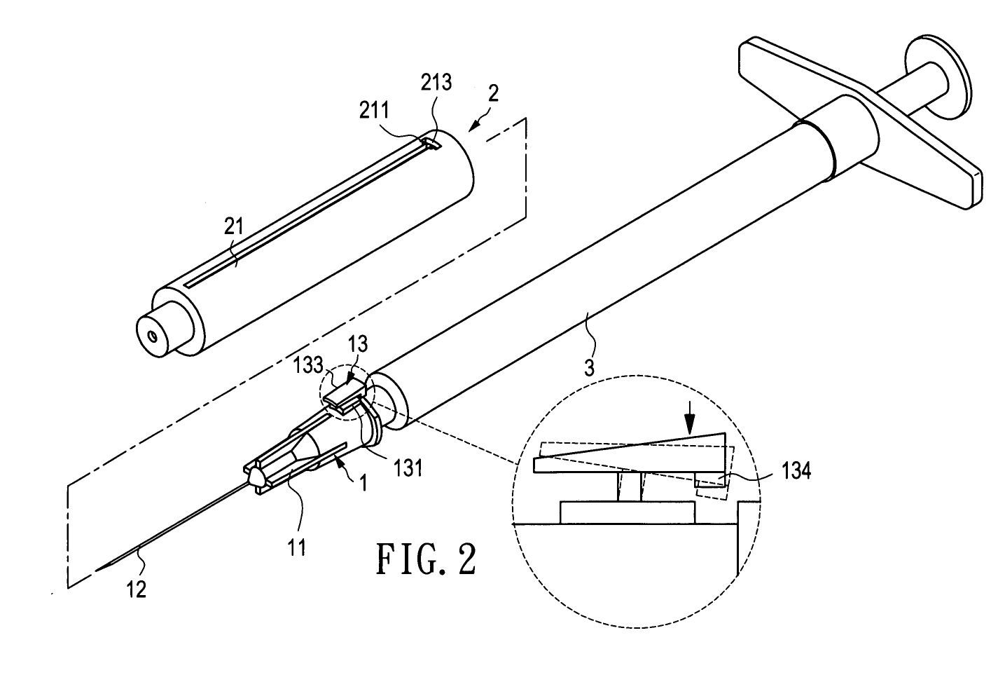

Referring to FIG. 2, in the single-use safety injector structure provided by the invention, the outer periphery of the protective cover 2 is provided with no salient 211 and no non-return stopping block 212. The gliding track 21 is concavely provided with an opening 213 at the terminal thereof. One or more salients 211 are convexly provided between the gliding track 21 and the opening 213. The upper stopping portion 133 of the gliding block 13 on the terminal and lower portion thereof is provided with a raised block 134 to be accommodated within the opening 213. Before the single-use safety injector structure provided by the invention is used, the upper portion of the raised block 134 of the upper stopping portion 133 is sustained against the outer periphery of the protective cover 2, and the rear wall of the raised block 134 is pushed against the front end of the salient 211. When the single-use safety injector structure provided by the invention is to be used, one thing to be done is to press the front end of the upper stopping portion 133. By using the guiding rod 132 as a lever, the raised block 134 on the terminal and lower portion of the upper stopping portion 133 is pulled upwardly. At that time, the protective cover 2 can be pulled backwardly to expose the injection needle 12 from the front end of the protective cover 2, and thus the injecting or drawing-out action is performed. After the injecting or drawing-out action is completed, the front end of the upper stopping portion 133 is pressed again. By using the guiding rod 132 as a lever, the raised block 134 on the terminal and lower portion of the upper stopping portion 133 is pulled upwardly. As such, the raised block 134 of the upper stopping portion 133 passes through the salient 211, and accommodated within the opening 213 at the rear end of the protective cover 2. Meanwhile, even if the front end of the upper stopping portion 133 is pressed again, the raised block 134 fails to escape from the opening 213. Accordingly, the single-use safety injector structure of the present invention can be operated without worrying about its repeated use.

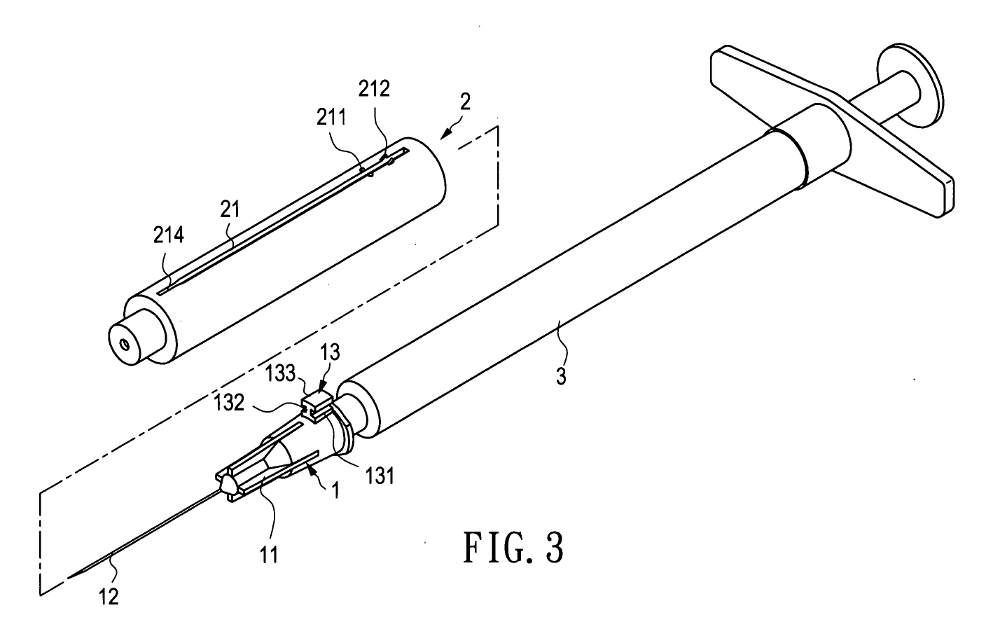

Referring to FIG. 3, in the single-use safety injector structure provided by the invention, the gliding track 21 of the protective cover 2 in the vicinity of the front end thereof is provided with no salient 211, and the front end of the gliding track 21 forms a tapered clip portion 214. As such, when the protective cover 2 is pushed backwardly to perform the injecting or drawing-out action, by using the clip portion 214 to clip both wall of the guiding rod 132, the protective cover 2 is fixed on the rear end of the syringe needle 1 so as to prevent the protective cover 2 from moving forwardly and hindering the injecting or drawing-out action.

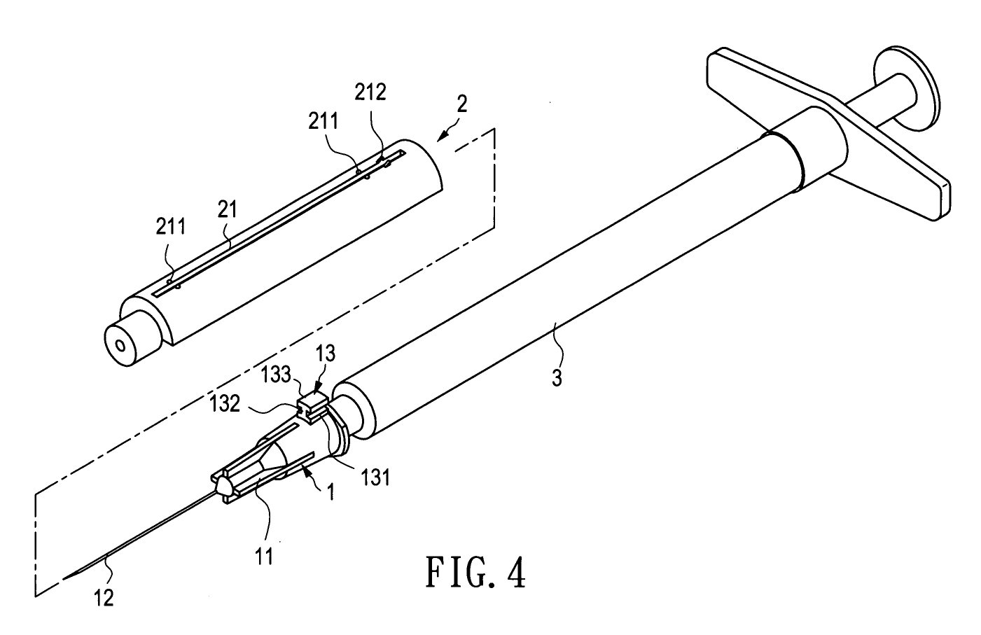

Referring to FIG. 4, in the single-use safety injector structure provided by the invention, the protective cover 2 remains the front portion for encapsulating the tip of the injection needle 12, but the rear portion thereof can be replaced with a sheet or semi-circular body so as to avoid sheltering graduation on the injector. As such, when a blood sample is to be quantitatively drawn out or a drug is to be quantitatively injected, the problem of excessively or insufficiently drawing-out or injecting can be avoided.

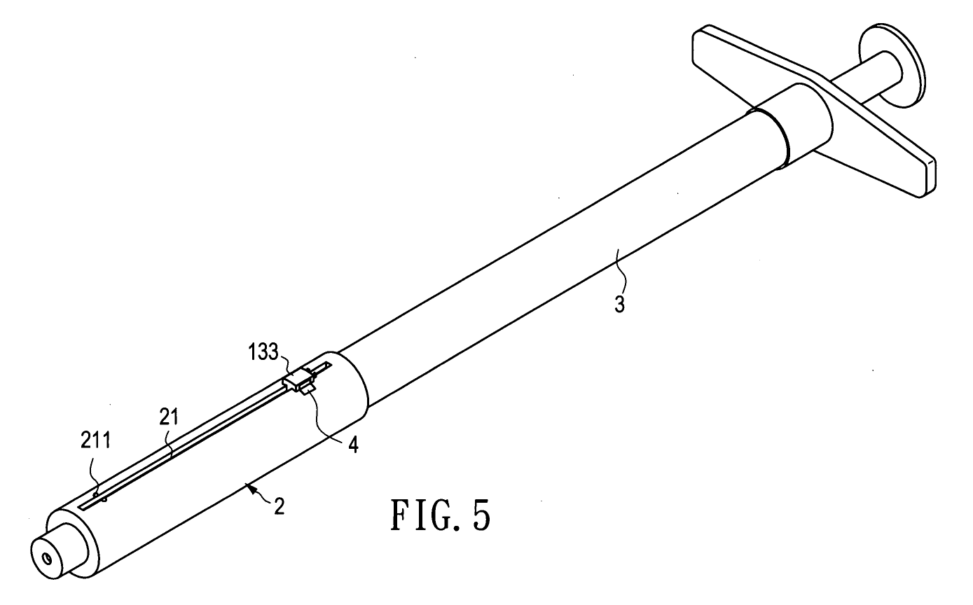

Referring to FIG. 5, in the single-use safety injector structure provided by the invention, the gliding track 21 in the vicinity of the rear end thereof is provided with no salient 211 but directly connected to the upper stopping portion 133 and the protective cover 2 via a connection member 4. When the single-use safety injector structure provided by the invention is to be used, the only thing to be done is to tear open the connection member 4. As such, the protective cover 2 is pushed backwardly to perform the injecting or drawing-out action. Alternatively, the connection member 4 can be replaced by viscose or other adhesive element.

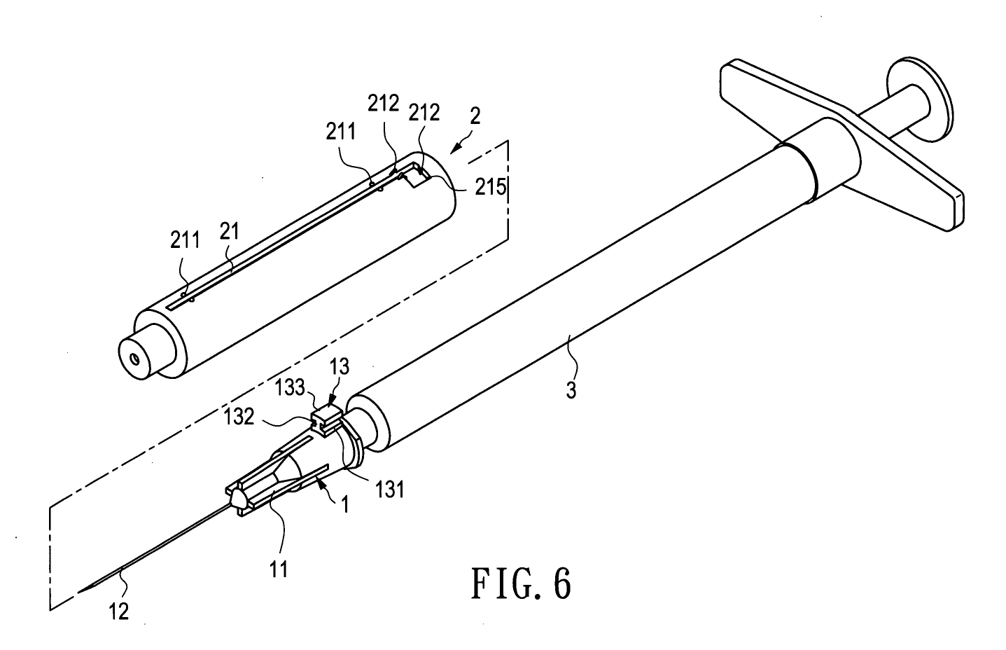

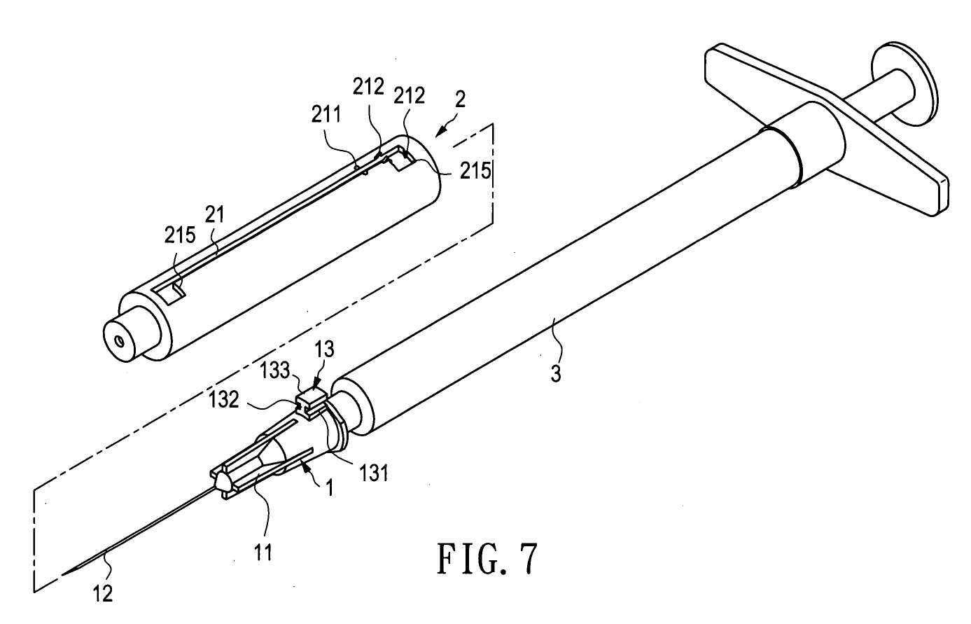

Referring to FIGS. 6 and 7, in the single-use safety injector structure provided by the invention, the protective cover 2 can be also provided with a whirl slot 215 behind one side of the rear-end non-return stopping block 212. Several non-return stopping blocks 212 are provided on the connection portion between the whirl slot 215 and the gliding track 21. As such, after the injecting or drawing-out action is completed, the protective cover 2 can be firstly pushed forwardly to the front end of the syringe needle 1. Then, the protective cover 2 is rotated such that the guiding rod 132 of the gliding block 13 is accommodated into the whirl slot 215. In addition, the guiding rod 132 fails to escape from the whirl slot 215 because it is stopped by the non-return stopping block 212. Accordingly, the single-use safety injector structure of the present invention can be operated without worrying about its repeated use. Alternatively, the non-return stopping block 212 in the vicinity of the front end thereof can be also provided with no salient 211, and one side on the front end of the gliding track 21 is provided with a whirl slot 215. As such, before the injecting or drawing-out action is performed, when the protective cover 2 is pulled backwardly, by rotating the protective cover 2, the guiding rod 132 can be accommodated into the whirl slot 215 so as to prevent the protective cover 2 from moving forwardly and hindering the injecting or drawing-out action. After the injecting or drawing-out action is completed, the only thing to be done is to rotate the protective cover 2 again. As such, the guiding rod 132 is escaped from the whirl slot 215 and then the protective cover 2 is pushed forwardly.

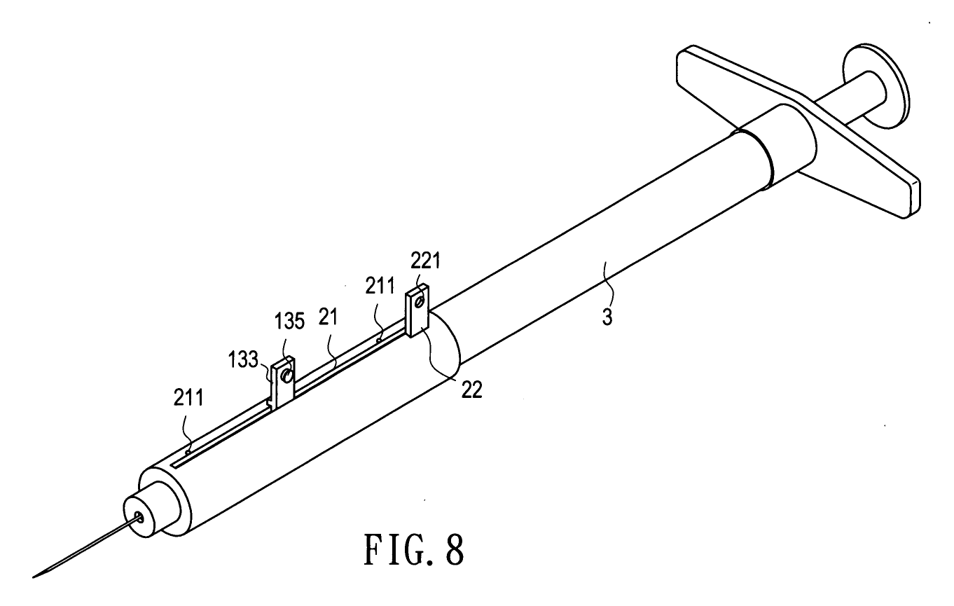

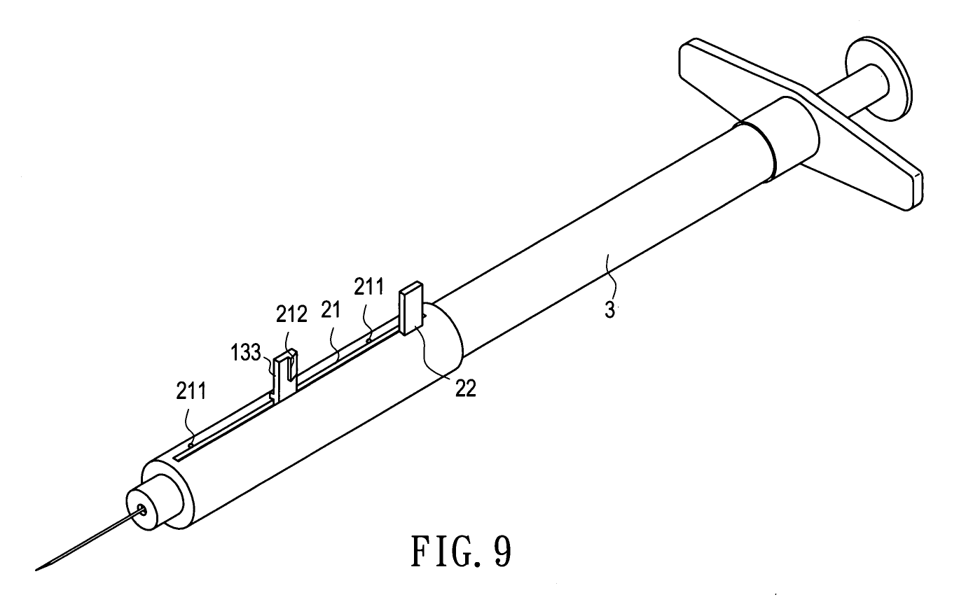

Referring to FIGS. 8 and 9, in the single-use safety injector structure provided by the invention, the protective cover 2 can be also provided with no non-return stopping block 212 on the rear end of the gliding track 21, but a raised block 22 is extended from one side at the rear end of the gliding track 21. A fastening slot 221 is provided on the raised block 22. The upper stopping portion 133 is provided with a protruding buckle 135 at a position corresponding to the fastening slot 221 of the raised block 22. After the single-use safety injector structure provided by the invention completes the injecting or drawing-out action, the only thing to be done is to buckle the protruding buckle 135 into the fastening slot 221. Accordingly, the single-use safety injector structure of the present invention can be operated without worrying about its repeated use. Alternatively, the position of the fastening slot 221 on the raised block 22 can be exchanged with the position of the protruding buckle 135 on the upper stopping portion 133. In addition, the raised block 22 can be provided at a position near the rear end of the gliding track 21, no fastening slot 221 is provided on the raised block 22, and the protruding buckle 135 provided on the upper stopping portion 133 is replaced by a non-return stopping block 212. After the injecting or drawing-out action is completed, when the protective cover 2 is pushed forwardly such that the upper stopping portion 133 is moved backwardly to the rear portion of the raised block 22. As such, the protective cover 2 fails to be pushed backwardly again since it is stopped on the rear-end wall of the raised block 22 by means of the non-return stopping blocks 212. Accordingly, the single-use safety injector structure of the present invention can be operated without worrying about its repeated use. Alternatively, the non-return stopping block 212 on the upper stopping portion 133 can also be provided on the raised block 22.

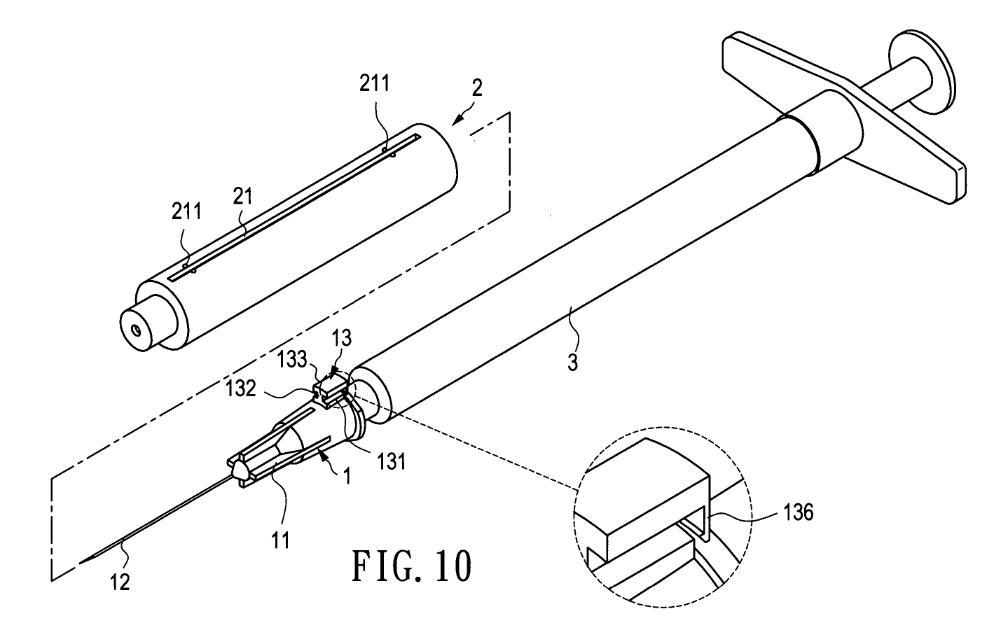

Referring to FIG. 10, in the single-use safety injector structure provided by the invention, no non-return stopping block 212 is provided on the rear end of the gliding track 21. The terminal and lower portion of the upper stopping portion 133 extends downwardly a fastening hook 136 to be hooked onto the rear-end inner wall of the protective cover 2. After the single-use safety injector structure provided by the invention completes the injecting or drawing-out action, when the protective cover 2 is pushed forwardly to the tip of the syringe needle 1, the fastening hook 136 will pass through the rear edge of the protective cover 2 and be hooked onto the rear-end inner wall of the protective cover 2. As such, the protective cover 2 fails to be pushed backwardly again.

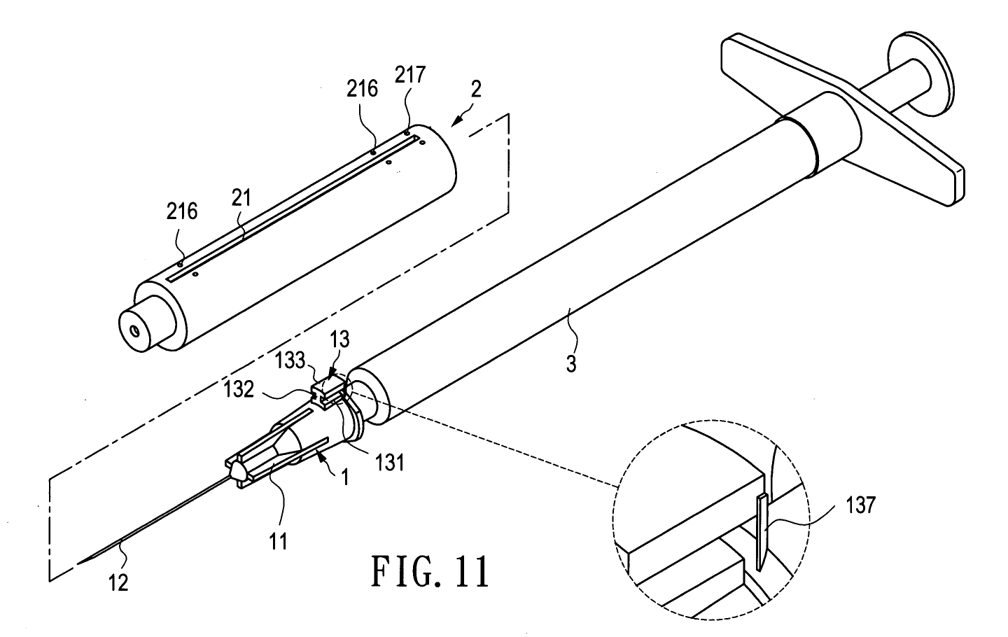

Referring to FIG. 11, in the single-use safety injector structure provided by the invention, one or both sides of the upper stopping portion 133 extend downwardly a penetrating hook 137, one or both sides of the gliding track 21 of the protective cover 2 in the vicinity of the front end and the rear end thereof are provided with several recesses 216, and one or both sides at the terminal of the gliding track 21 of the protective cover 2 are provided with a penetrating hook 137. Before the single-use safety injector structure provided by the invention is used, the penetrating hook 137 is retained in the recess 216 near the rear end of the gliding track 21. When the single-use safety injector structure provided by the invention is used, the protective cover 2 is pulled backwardly such that the penetrating hook 137 is detached from the recess 216 near the rear end of the gliding track 21 and placed into the recess 216 near the front end of the gliding track 21. As such, the difficulty of the drawing-out or injecting action resulting from the forward movement of the protective cover 2 can be avoided. After the drawing-out or injecting action is completed, the only thing to be done is to push the protective cover 2 forwardly. As such, the penetrating hook 137 will drop into a penetrating hole 217 at the terminal of the gliding track 21, and thus the protective cover 2 fails to move backwardly. Accordingly, the single-use safety injector structure of the present invention can be operated without worrying about its repeated use.

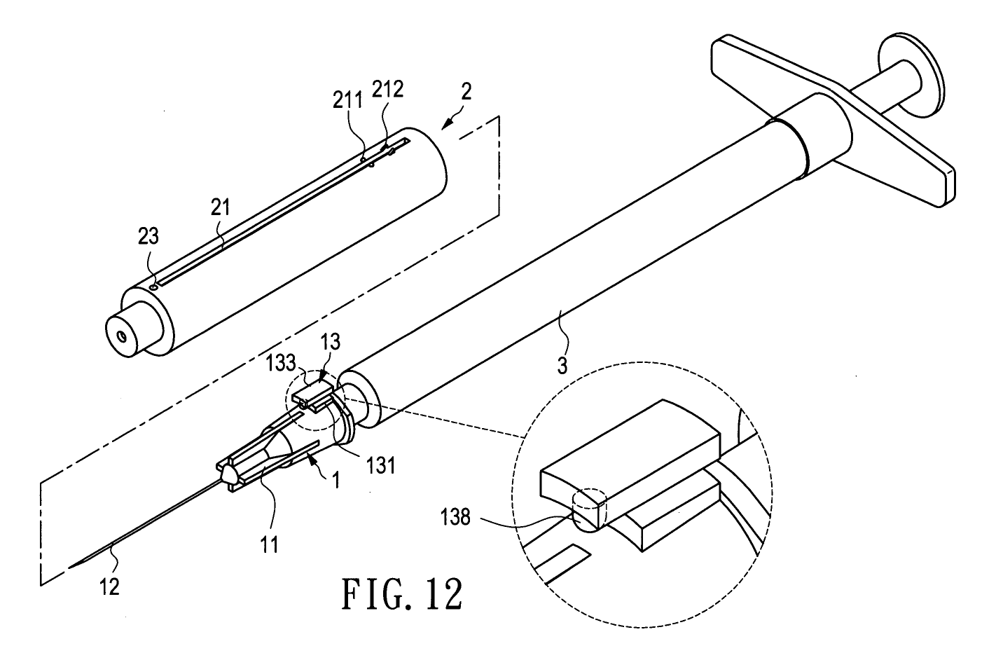

Referring to FIG. 12, in the single-use safety injector structure provided by the invention, the gliding track 21 in the vicinity of the front end thereof can be provided with no salient 211. The protective cover 2 is provided with an indenting buckle 23 on the front end of the gliding track 21, and the front end and lower edge of the upper stopping portion 133 is provided with a protruding buckle 138. When the single-use safety injector structure provided by the invention is used, the protective cover 2 is pulled backwardly such that the protruding buckle 138 of the upper stopping portion 133 is engaged with the indenting buckle 23 of the protective cover 2. As such, the difficulty of the drawing-out or injecting action resulting from the forward movement of the protective cover 2 can be avoided. Alternatively, the position of the protruding buckle 138 of the upper stopping portion 133 can be exchanged with the position of the indenting buckle 23 of the protective cover 2.

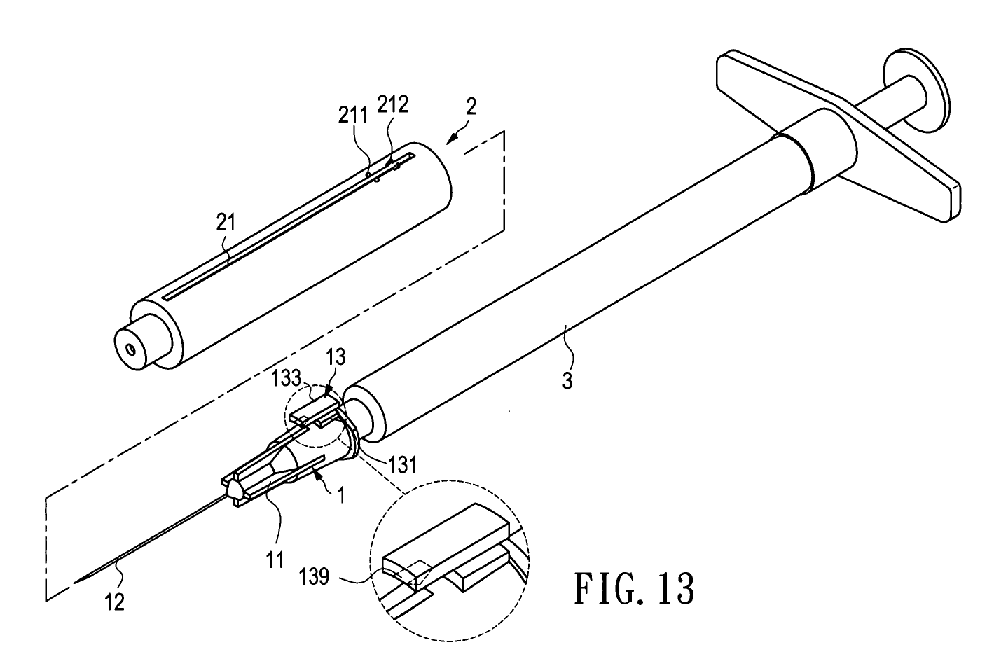

Referring to FIG. 13, in the single-use safety injector structure provided by the invention, the gliding track 21 in the vicinity of the front end thereof can be provided with no salient 211. The front end and lower edge of the upper stopping portion 133 is provided with a protruding portion 139. Each of the front and rear ends of the protruding portion 139 comprises an inclined plane. When the single-use safety injector structure provided by the invention is to be used and the protective cover 2 is pulled backwardly, by means of the front-end inclined plane, the protruding portion 139 is detached from the gliding track 21 and then moved to the front edge of the protective cover 2. When the injecting or drawing-out action is done, by using the protruding portion 139 to stop on the front-end wall of the protective cover 2, the protective cover 2 is prevented from moving. After the drawing-out or injecting action is completed, the only thing to be done is to push the protective cover forwardly. Meanwhile, by means of the rear-end inclined plane, the protruding portion 139 can be detached from the front-end wall of the protective cover 2, and enter the gliding track 21 again.

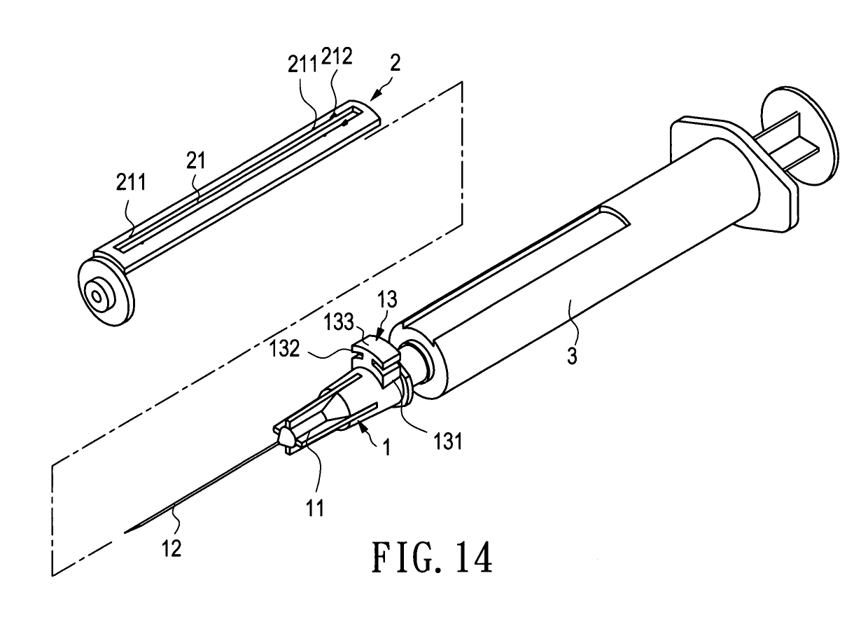

Referring to FIG. 14, in the single-use safety injector structure provided by the invention, the protective cover 2 remains the front portion for encapsulating the tip of the injection needle 12, but the rear portion thereof can be replaced with a sheet body 24. In addition, the syringe 3 is provided with an indenting portion 31 at a position corresponding to the sheet body on the rear portion of the protective cover 2. As such, when the protective cover 2 is pushed backwardly, the sheet body 24 on the rear portion of the protective cover 2 will be introduced into the indenting portion 31 of the syringe 3.

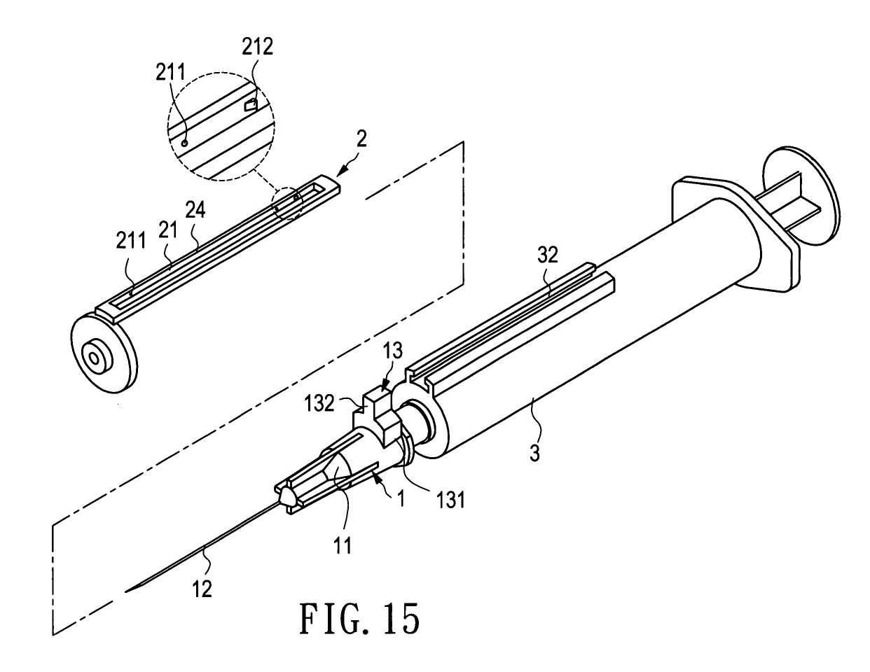

Referring to FIG. 15, in the single-use safety injector structure provided by the invention, the protective cover 2 remains the front portion for encapsulating the tip of the injection needle 12, but the rear portion thereof can be replaced with a sheet body 24. In addition, the salients 211 and the non-return stopping block 212 are provided on the inner wall of the gliding track 21, no upper stopping portion 133 is provided on the gliding block 13, and an encapsulating portion 32 for accommodating the sheet body 24 on the rear portion of the protective cover 2 is convexly provided on the outer periphery of the syringe 3.

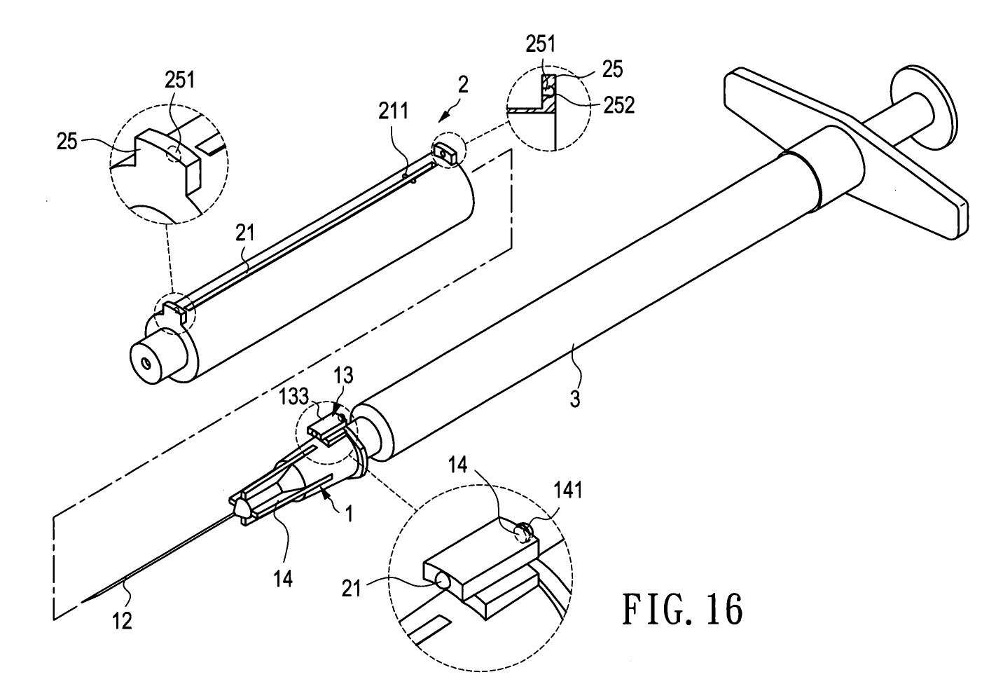

Referring to FIG. 16, in the single-use safety injector structure provided by the invention, the gliding track 21 in the vicinity of the front end thereof can be provided with no salient 211. The front edge of the upper stopping portion 13 is provided with a protruding buckle 14. The front end of the outer periphery of the protective cover 2 is convexly provided with a raised block 25. The raised block 25 is provided with an indenting buckle 251 at a position corresponding to the protruding buckle 14 at the front end of the outer periphery of the protective cover 2. As such, when the protective cover 2 is pulled backwardly, by the engagement of the protruding buckle 14 and the indenting buckle 251, the protective cover 2 is prevented from moving. Alternatively, the rear edge of the upper stopping portion 13 is provided with a protruding buckle 14, the terminal of the protruding buckle 14 is provided with a plugging portion 141, the terminal of the protective cover 2 is provided with a raised block 36 having an indenting buckle 251, and the inner side of the indenting buckle 251 is provided with a buckling portion 252. As such, after the single-use safety injector structure of the invention completes the injecting or drawing-out action, by pushing the protective cover 2 forwardly, the protruding buckle 14 at the rear edge of the upper stopping portion 13 is accommodated into the indenting buckle 251. In addition, by the engagement of the plugging portion 141 and the buckling portion 252, the protective cover 2 fails to be pushed backwardly again. Accordingly, the single-use safety injector structure of the present invention can be operated without worrying about its repeated use. Alternatively, the position of the protruding buckle 14 on the front or rear end of the upper stopping portion 13 can be exchanged with the position of the indenting buckle 251 on the raised block 25.

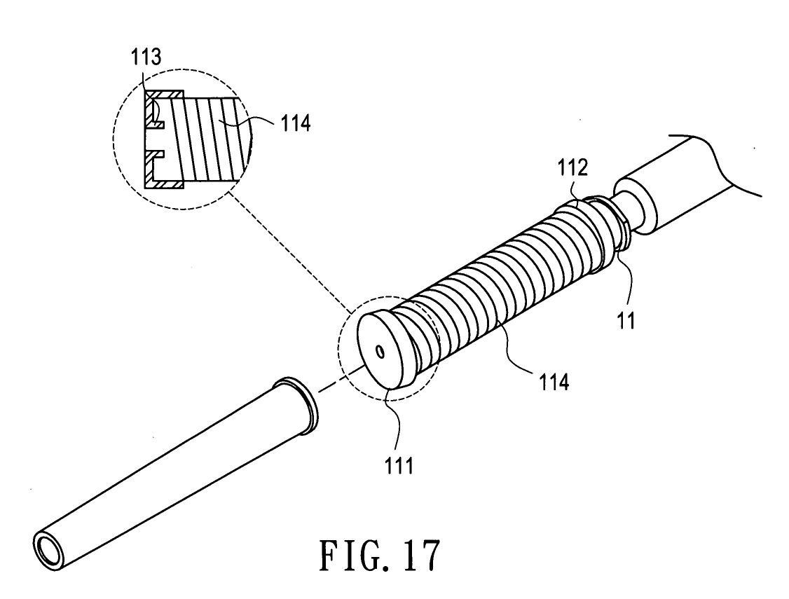

Referring to FIG. 17, in the single-use safety injector structure provided by the invention, the insertion portion 11 can be provided with no gliding block 13. The protective cover 2 is consisted of a front protective cover 111 and a rear protective cover 112. The periphery of the open side of the front protective cover 111 is provided with a ring stopping portion 113. The front protective cover 111 is connected to the rear protective cover 112 via a ring-shaped sheet 114. The axel center of the ring-shaped sheet 114 has a bias angle in respect with the axel center of the front protective cover 111 and the rear protective cover 112. Before the single-use safety injector structure provided by the invention is used, the front protective cover 111 and the rear protective cover 112 are both sleeved around the outer periphery of the insertion portion 11 of the syringe needle 1, and the injection needle 12 is covered by a conventional syringe cover. When a user is ready to perform the drawing-out or injecting action, the only thing to be done is to remove the conventional syringe cover. After the drawing-out or injecting action is completed, the front protective cover 111 is pulled forwardly. Meanwhile, since the axel center of the ring-shaped sheet 114 has a bias angle in respect with the axel center of the front protective cover 111 and the rear protective cover 112, the front protective cover 111 will be shifted upon being pulled. As such, the injection needle 12 is accommodated between the inner wall of the front protective cover 111 and the ring stopping portion 113, and the injection needle 12 fails to be pierced through the front protective cover 111. Accordingly, the single-use safety injector structure of the present invention can be operated without worrying about its repeated use. Alternatively, the axel center of the ring-shaped sheet 114 can be coaxial with the axel centers of the front protective cover 111 and the rear protective cover 112. However, the ring-shaped sheet 114 is connected to the front protective cover 111 via a single-point or single-side connection manner such that a biasing condition occurs when the front protective cover 111 is pulled forwardly.

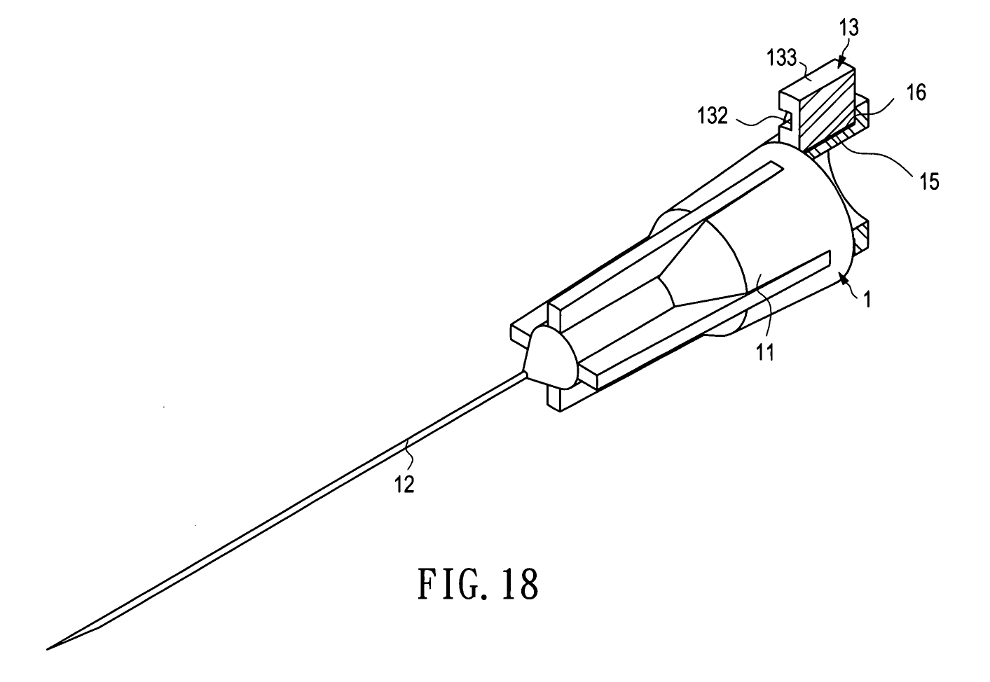

Referring to FIG. 18, in the single-use safety injector structure provided by the invention, the gliding block 13 could be separated from the insertion portion 11. The insertion portion 11 is concavely disposed with a ring-shape recess 15, a fastening hole 16 is provided in the center of the base 131 of the gliding block 13, and the fastening hole 16 is disposed around the outer periphery of the ring-shape recess 15. As such, when the single-use safety injector structure provided by the invention is used, the direction of the inclined plane at the tip of the injection needle 12 can be adjusted by rotating the insertion portion 11. Alternatively, the outer periphery of the front end of the insertion portion 11 corresponding to the inclined plane at the tip of the injection needle 12 can be provided with some marks. As such, after the injection needle 12 is inserted into the injecting site of the subject, the medical practitioner can realize the direction of the inclined plane at the tip of the injection needle 12 by rotating the insertion portion 11.

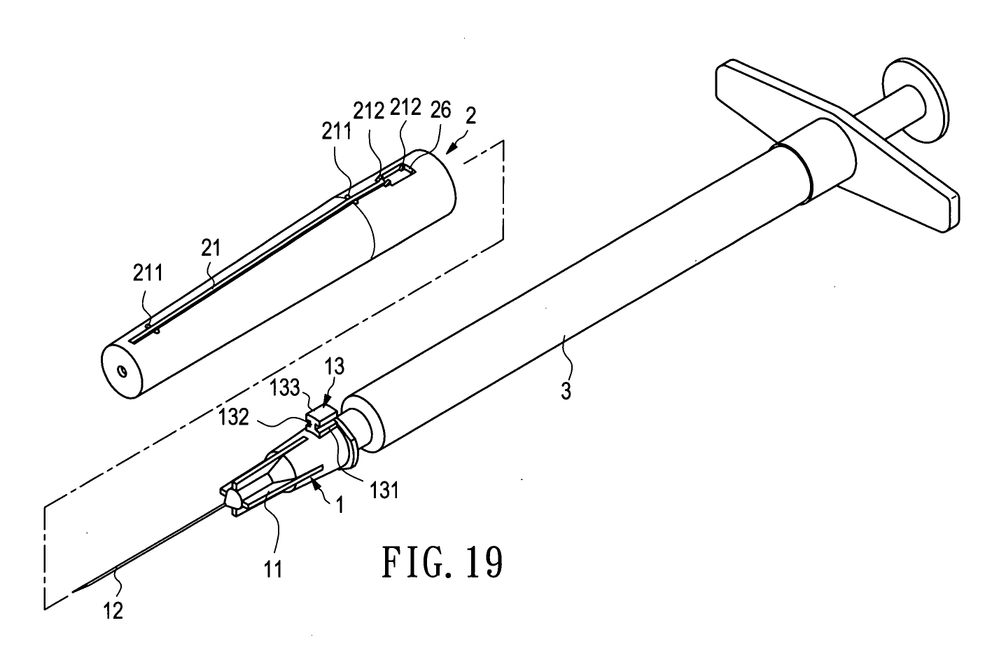

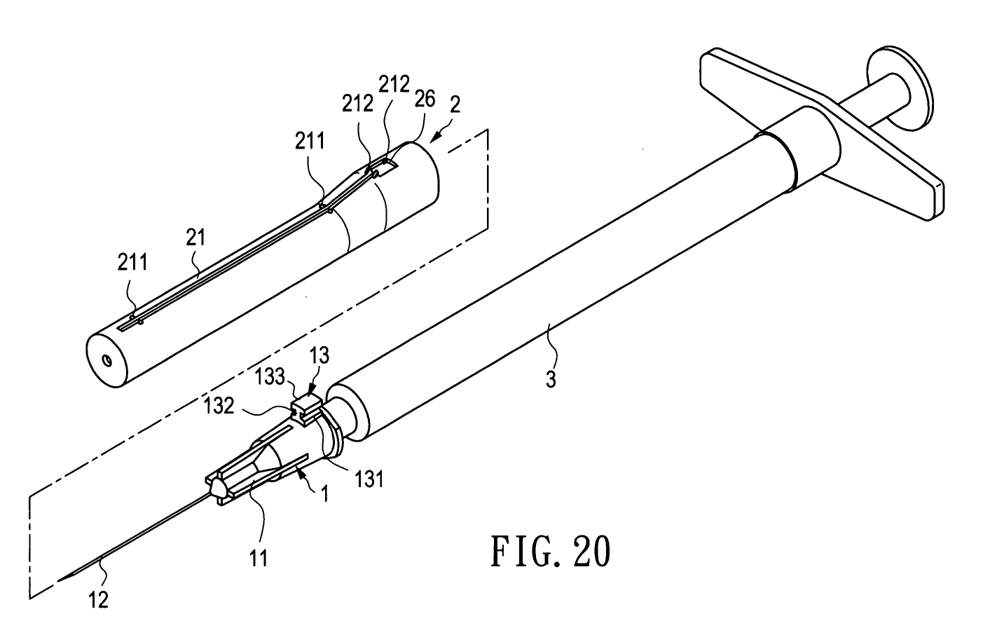

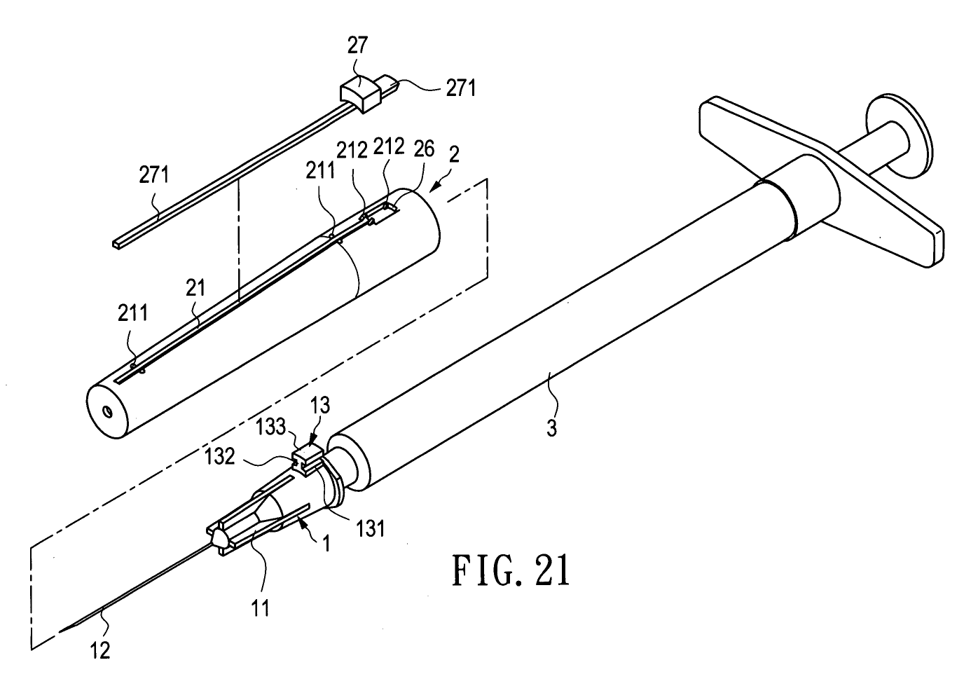

Referring to FIGS. 19, 20 and 21, in the single-use safety injector structure provided by the invention, the rear end of the non-return stopping block 212 of the protective cover 2 can be provided with an accommodating portion 26. The inner periphery of the accommodating portion 26 can be provided with several non-return stopping blocks 212. The protective cover 2 is tapered from the rear-end salient 211 of the gliding track 21 to the front end thereof. After the drawing-out or injecting action is completed and the protective cover 2 is pulled forwardly, the upper stopping portion 133 can be pressed into the accommodating portion 26. In addition, the upper stopping portion 133 fails to escape from the accommodating portion 26 because it is stopped by the non-return stopping block 212. Accordingly, the single-use safety injector structure of the present invention can be operated without worrying about its repeated use. Alternatively, the protective cover 2 is not tapered from the rear-end salient 211 of the gliding track 21 to the front end thereof. Instead, the protective cover 2 is raised from the rear-end salient 211 of the gliding track 21. In addition, no rear-end salient 211 is provided on the gliding track 21, but a cap sheath 27 is covered on the upper stopping portion 133. Each of the front and the rear ends of the cap sheath 27 extends a cap body 271 to be placed into the gliding track 21 and the accommodating portion 26. As such, before the single-use safety injector structure provided by the invention is used, by the fixture of the cap sheath 27, the protective cover 2 fails to move and thus the associated risk is avoided.

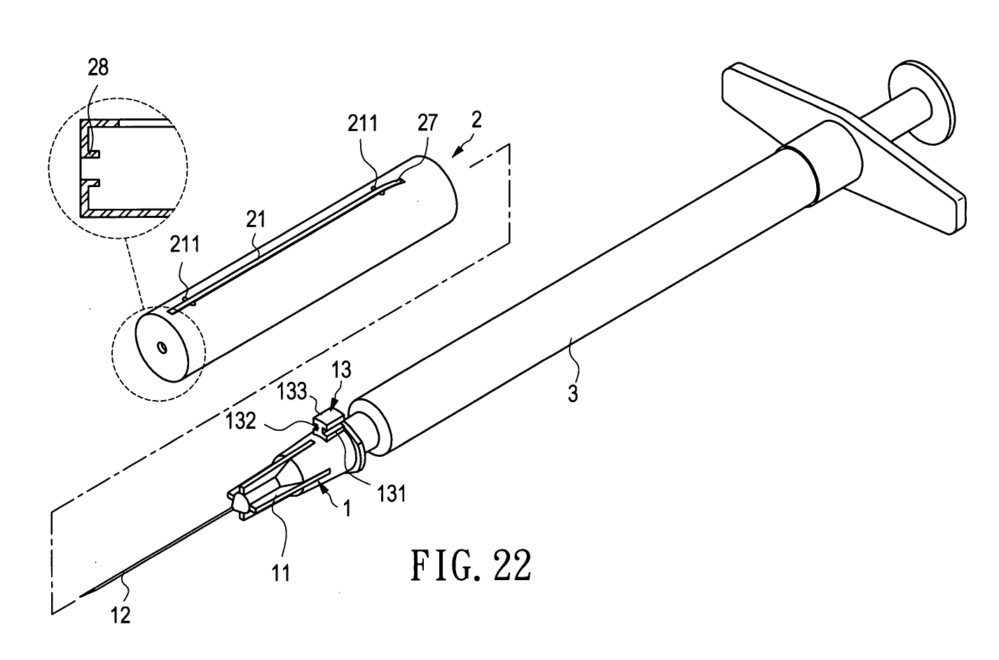

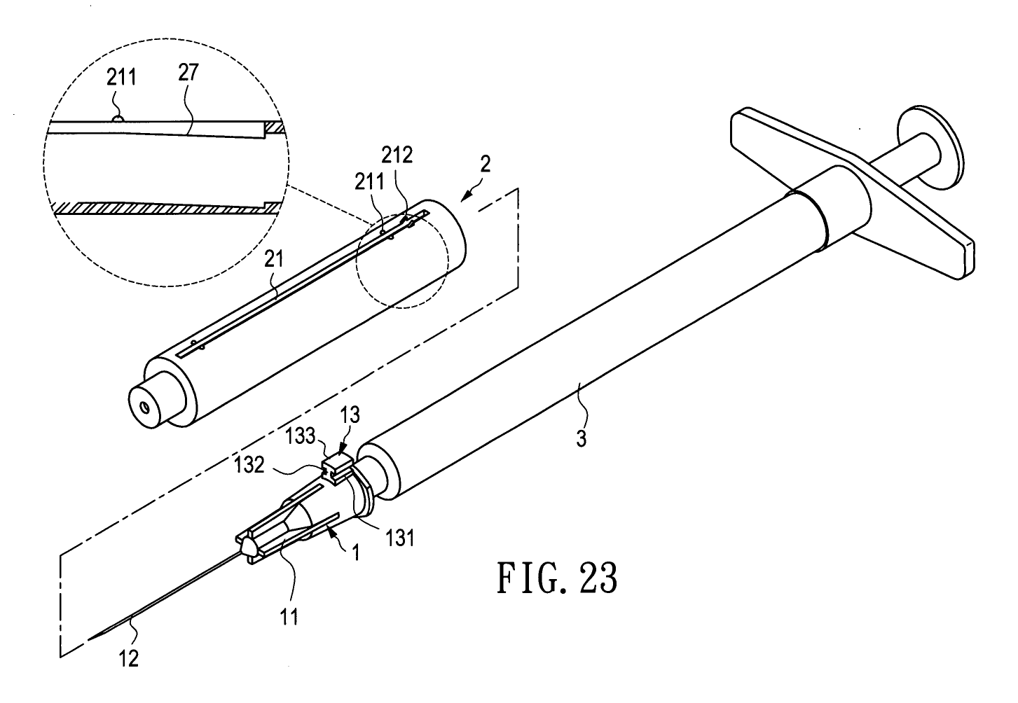

Referring to FIGS. 22 and 23, in the single-use safety injector structure provided by the invention, the rear end of the gliding track 21 is provided with no non-return stopping block 212. In the inner wall at the rear end of the gliding track 21 or the rear end of the protective cover 2, a bias oblique angle 27 is formed. The front end of the protective cover 2 is provided with a ring stopping portion 28 for the injection needle 12 to be pierced therethrough. After the injecting or drawing-out action is completed and the protective cover 2 is pulled forwardly, the protective cover 2 is inclined because the protective cover 2 is influenced by the bias oblique angle 27. As such, since the injection needle 12 is stopped by the ring stopping portion 28, the injection needle 12 fails to be pierced through the protective cover 2. Accordingly, the single-use safety injector structure of the present invention can be operated without worrying about its repeated use.

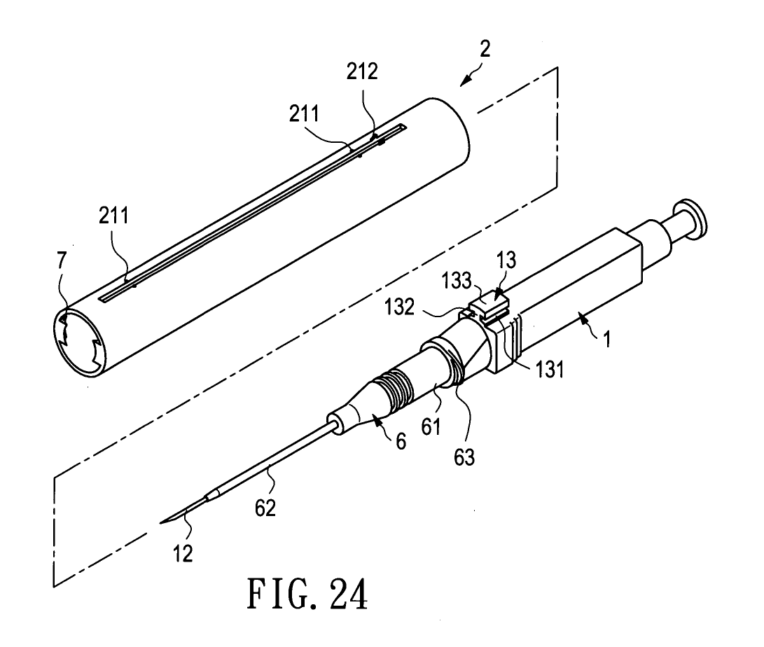

Referring to FIG. 24, the single-use safety injector structure provided by the invention is consisted essentially of:

a syringe needle 1, having an injection needle 12 inserted into the front end thereof, wherein the outer periphery of the syringe needle 1 is provided with a gliding block 13, and the gliding block 13 includes a base 131, a guiding rod 132 and an upper stopping portion 133;

a catheter 6, comprising a sheathing tube 61 provided with a flexible tube 62 extended from the front end thereof and a ring stopping portion 63 at the rear periphery thereof; and

a protective cover 2, provided with a gliding track 21 on the outer periphery thereof, wherein one or both sides in the vicinity of the front end and rear end of the gliding track 21 are provided with salients 211, the gliding track 21 is provided with one or more non-return stopping blocks 212 behind and in the vicinity of the rear-end salient 211, and the inner periphery of the front-end open portion of the protective cover 2 is provided with several hooking portions 7.

The catheter 6 is connected to the front end of the syringe needle 1 via the sheathing tube 61. The outer portion of the injection needle 12 is covered by the flexible tube 62. The protective cover 2 is sleeved around the outer periphery of the syringe needle 1. By means of the guiding rod 132, the syringe needle 1 penetrates through the gliding track 21 of the protective cover 2. By means of the base 131, the syringe needle 1 is sustained against the inner wall of the protective cover 2. By means of the upper stopping portion 133, the syringe needle 1 is sustained against the external wall of the protective cover 2. Before the single-use safety injector structure provided by the invention is used, the upper stopping portion 133 is fixed between the rear-end salient 211 on the gliding track 21 of the protective cover 2 and the non-return stopping block 212. When the injection needle 12 is to be inserted into the injecting site of the subject, the only thing to be done is to pull the protective cover 2 backwardly. As such, the upper stopping portion 133 is detached from the rear-end salient 211 of the gliding track 21 and passes through the front-end salient 211 of the gliding track 21, such that the injection needle 12 is exposed from the front end of the protective cover 2. In addition, the rear-end wall of the upper stopping portion 133 of the gliding block 13 fails to move backwardly because it is stopped by the front-end salient 211 of the gliding track 21. Also, the hooking portion 7 at the front end of the protective cover 2 passes through the ring stopping portion 63 of the catheter 6. Meanwhile, the injection needle 12 of the syringe needle 1 can be inserted into the injecting site of the subject, and then the protective cover 2 is pushed forwardly. Since the hooking portion 7 of the protective cover 2 is sustained against the ring stopping portion 63 of the catheter 6, the flexible tube 62 of the catheter 6 can be subsequently pushed forwardly into the injecting site of the subject. At that moment, the medical practitioner presses down the connection portion between the flexible tube 62 of the catheter 6 and the injecting site of the subject, grasps the protective cover 2, and then pulls the syringe needle 1 backwardly. As such, the upper stopping portion 133 of the gliding block 13 will pass through the non-return stopping block 212 at the rear end of the gliding track 21, and the protective cover 2 fails to move backwardly again since it is stopped by means of the non-return stopping block 212. Furthermore, when the syringe needle 1 is pulled backwardly, the injection needle 12 will be drawn out from the injecting site of the subject. In addition, since the medical practitioner presses down the flexible tube 62 of the catheter 6, the catheter 6 will lie in the injecting site of the subject and be separated from the injection needle 12. At that moment, the medical practitioner can connect the sheathing tube 61 with an intravenous drip, an injector or other drawing equipment so as to perform the drawing-out or injecting action. Alternatively, the salient 211 and the non-return stopping blocks 212 arranged on the outer periphery of the protective cover 2 can be provided on the inner wall of the gliding track 21 so as to stop the guiding rod 132 of the gliding block 13. Furthermore, the salient 211 and the non-return stopping blocks 212 arranged on the outer periphery of the protective cover 2 can be provided on the inner wall of the gliding track 21 so as to stop the base 131 of the gliding block 13.

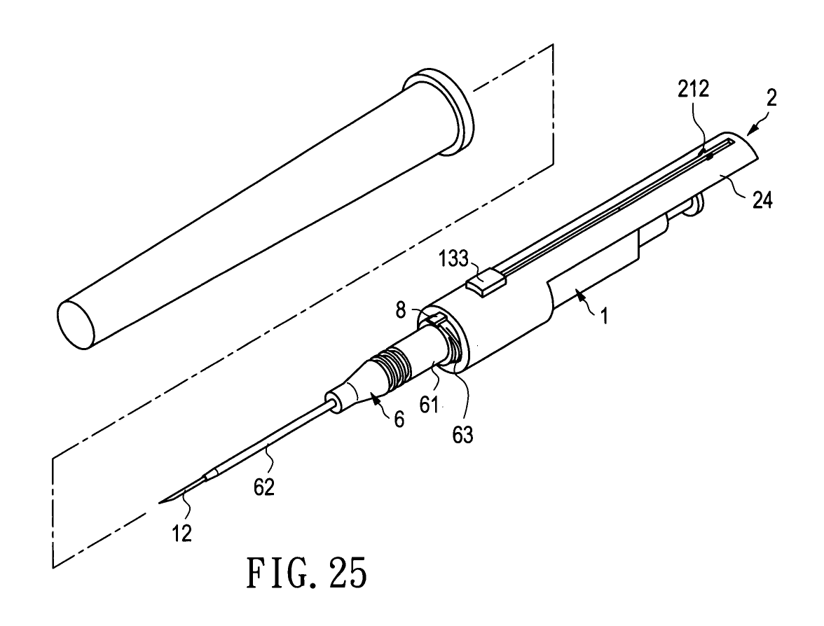

Referring to FIG. 25, in the single-use safety injector structure provided by the invention, the front edge of the protective cover 2 is sustained against the rear side of the sheathing tube 61. In addition, the front edge of the protective cover 2 is provided with several connection elements 8, e.g. buckle-hooks or viscose elements, so as to be hooked or bonded with the ring stopping portion 63 of the catheter 6. The injection needle 12 is covered by a conventional syringe cover. When a user is ready to perform the drawing-out or injecting action, the only thing to be done is to remove the conventional syringe cover. Then, the injection needle 12 of the syringe needle 1 is inserted into the injecting site of the subject, and the protective cover 2 is pulled forwardly. Since the front edge of the protective cover 2 is sustained against the ring stopping portion 63 of the catheter 6, the flexible tube 62 of the catheter 6 can be subsequently pushed forwardly into the injecting site of the subject. At that moment, the medical practitioner presses down the connection portion between the flexible tube 62 of the catheter 6 and the injecting site of the subject, grasps the protective cover 2, and then pulls the syringe needle 1 backwardly. As such, the upper stopping portion 133 of the gliding block 13 will pass through the non-return stopping block 212 at the rear end of the gliding track 21, and the protective cover 2 fails to move backwardly again since it is stopped by means of the non-return stopping block 212. Furthermore, when the syringe needle 1 is pulled backwardly, the injection needle 12 will be drawn out from the injecting site of the subject. In addition, since the medical practitioner presses down the flexible tube 62 of the catheter 6, the catheter 6 will lie in the injecting site of the subject and be separated from the injection needle 12. At that moment, the medical practitioner can connect the sheathing tube 61 with an intravenous drip, an injector or other drawing equipment so as to perform the drawing-out or injecting action. Alternatively, the protective cover 2 remains the front portion for encapsulating the tip of the injection needle 12, but the rear portion thereof is replaced with a sheet body 24.

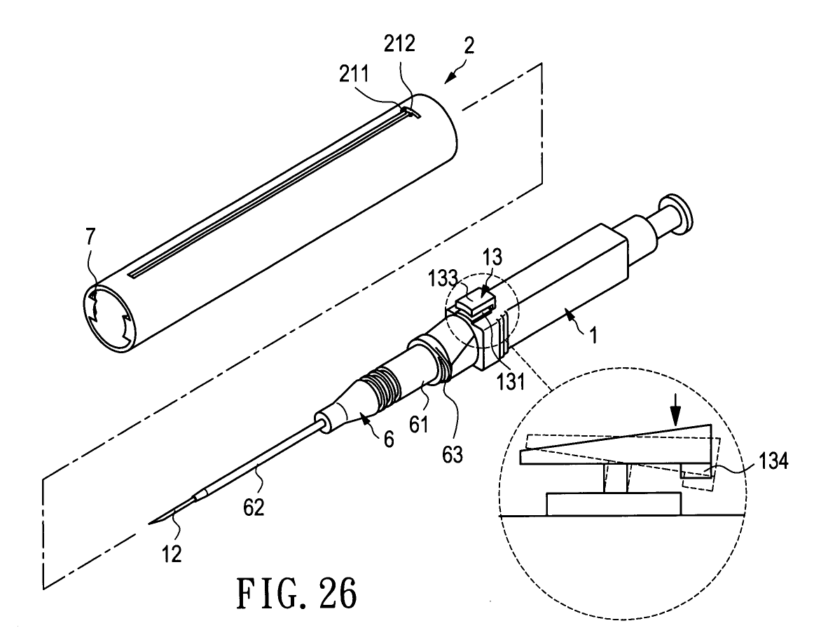

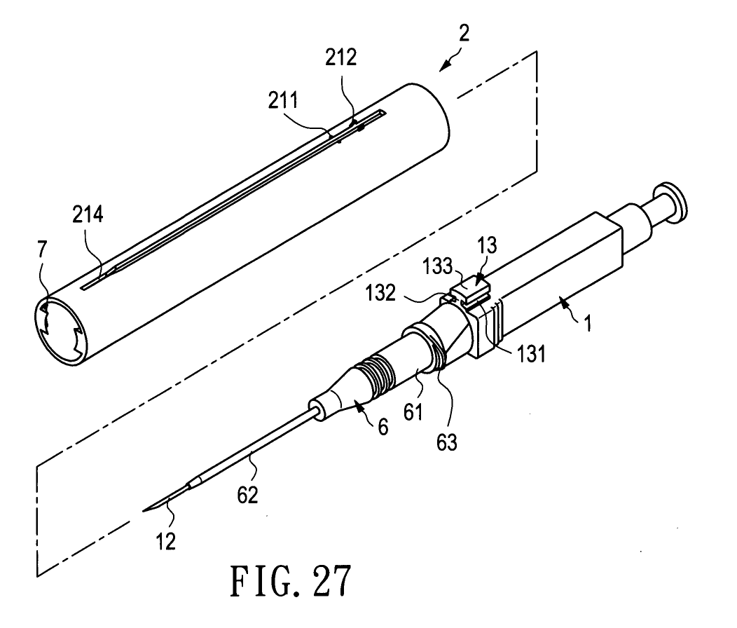

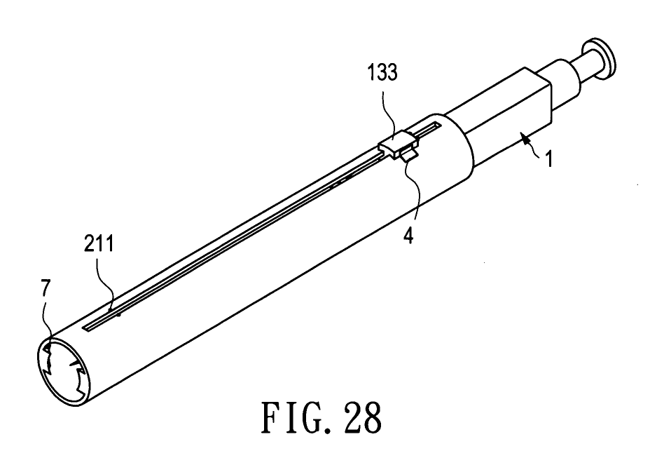

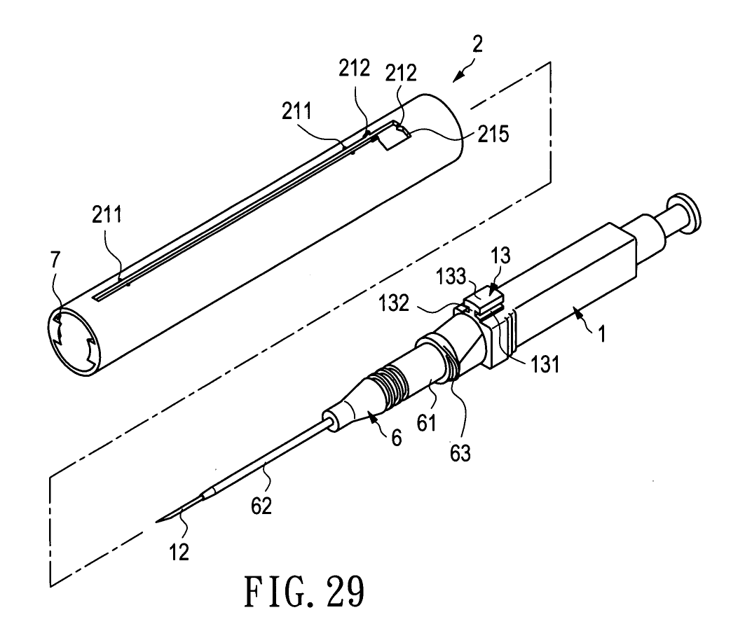

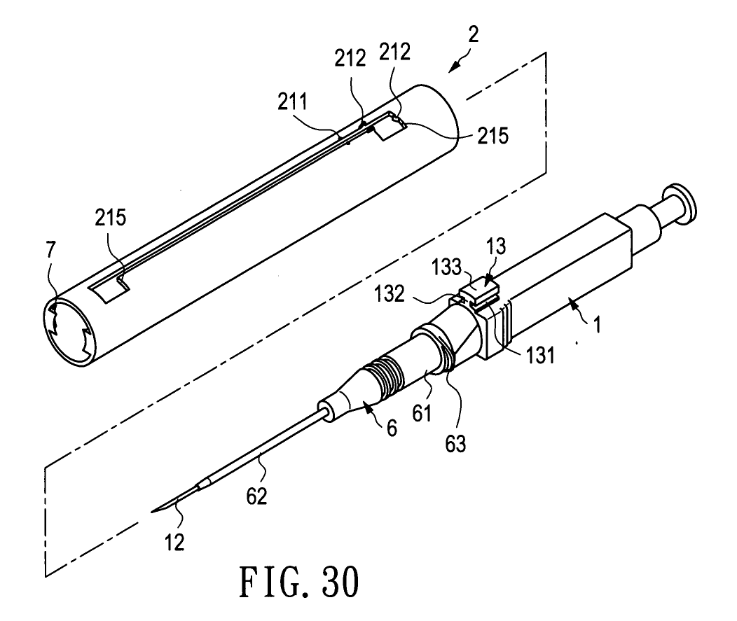

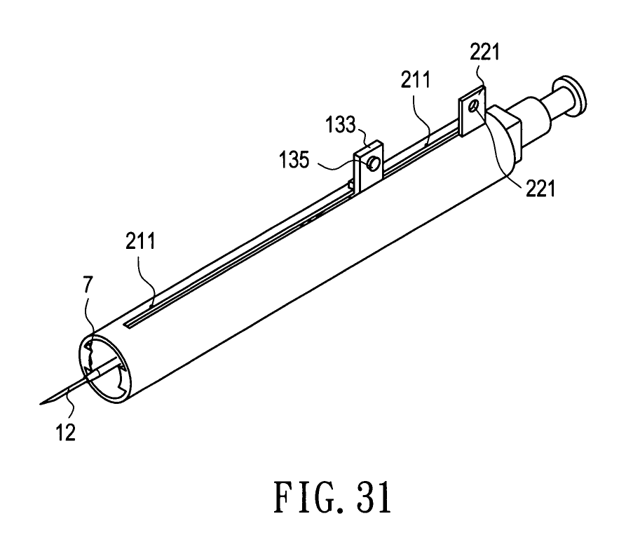

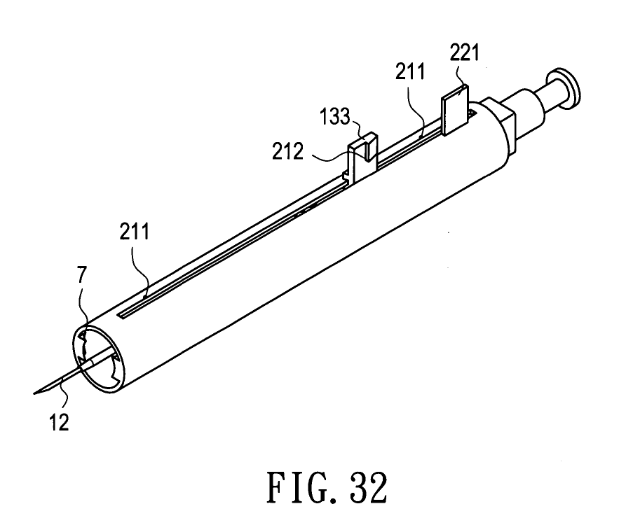

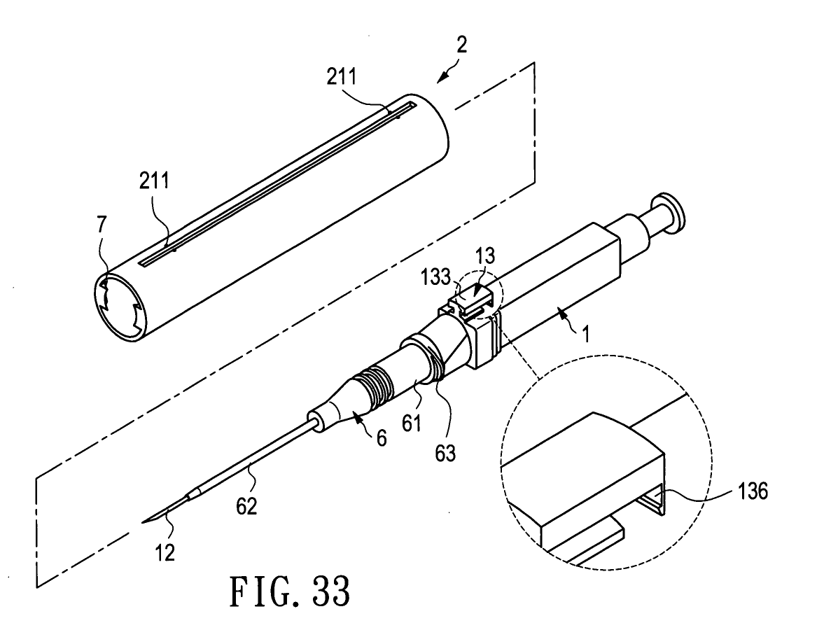

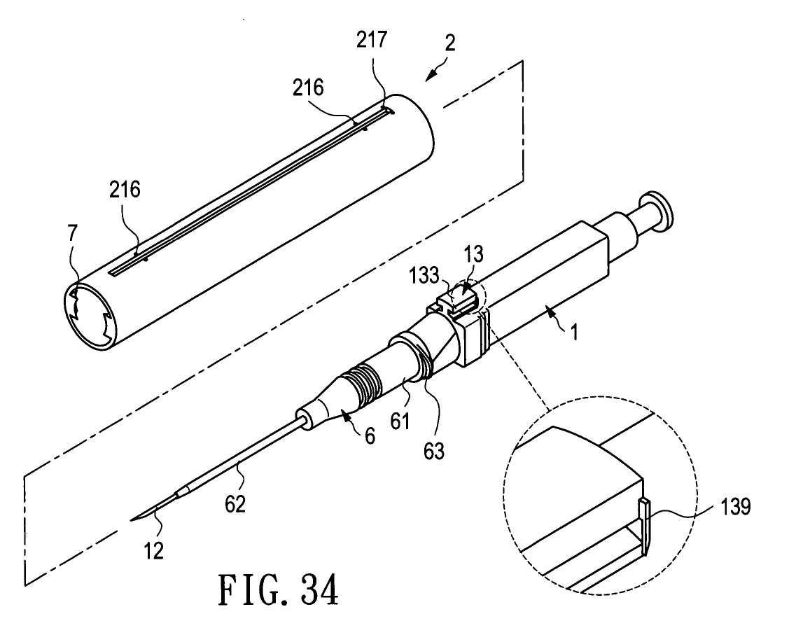

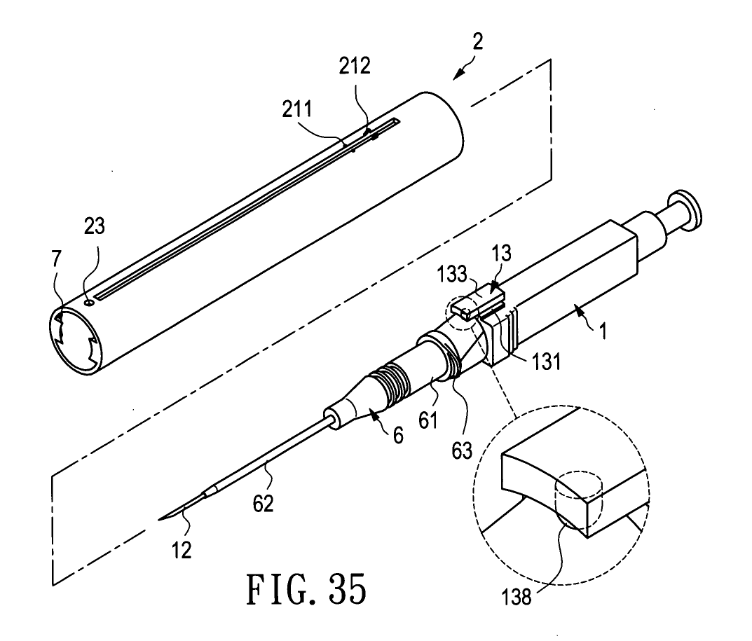

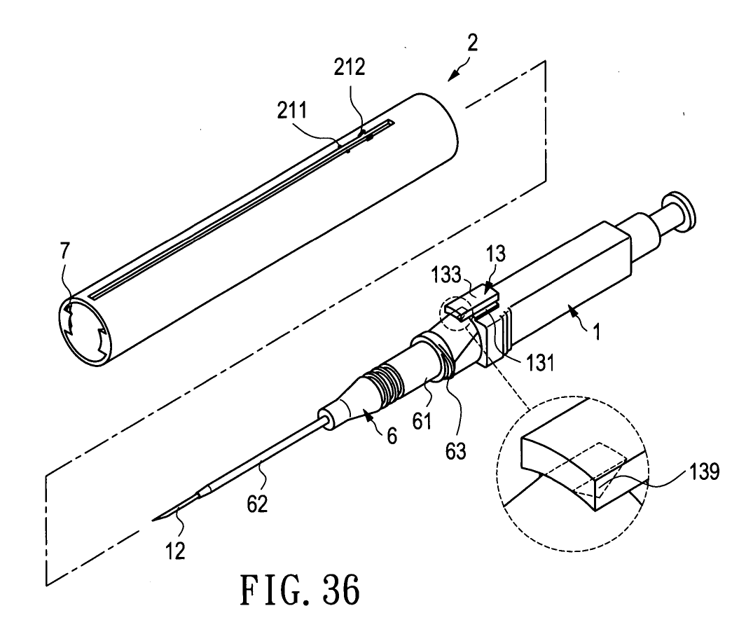

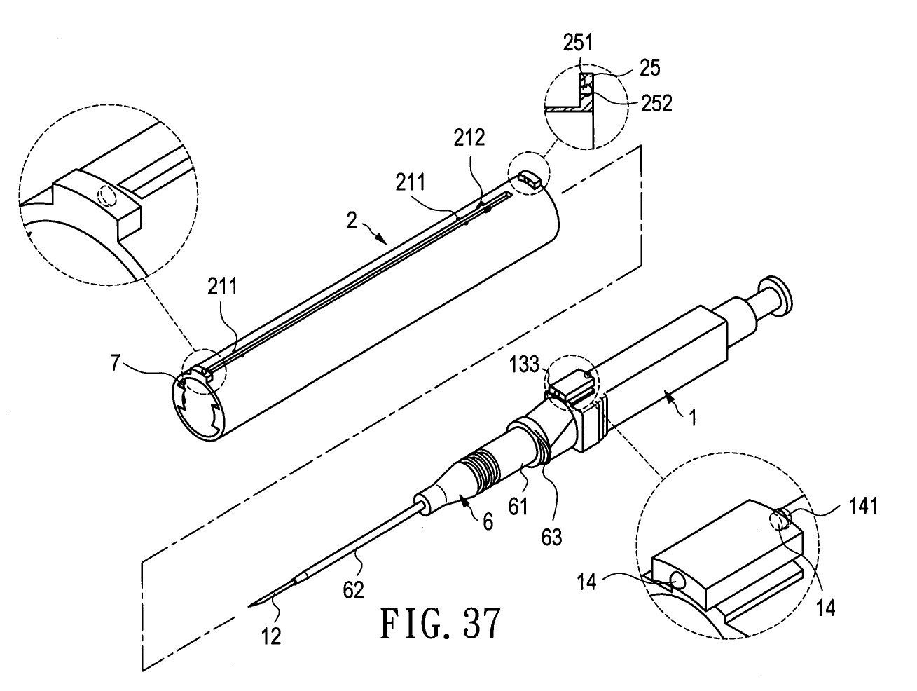

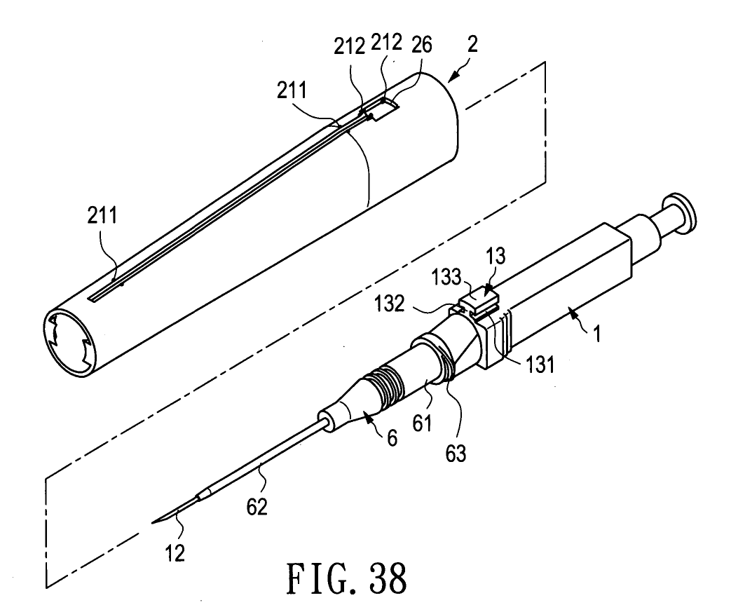

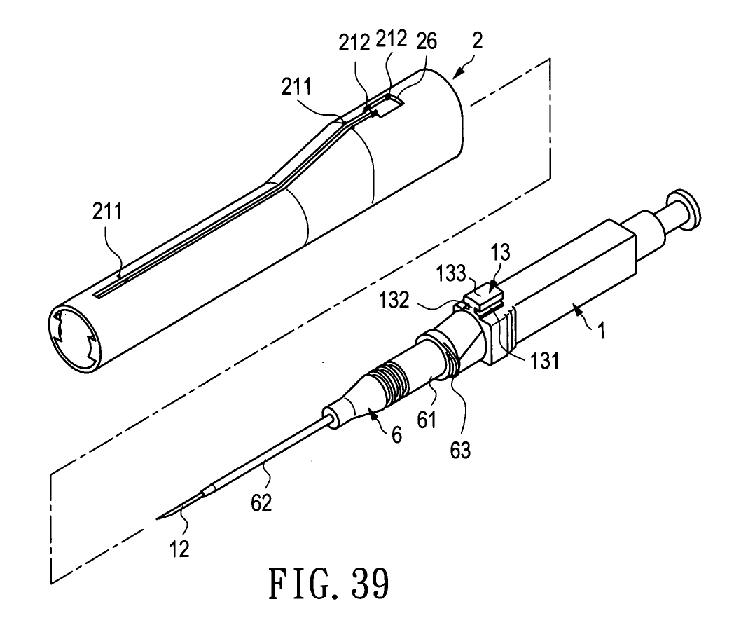

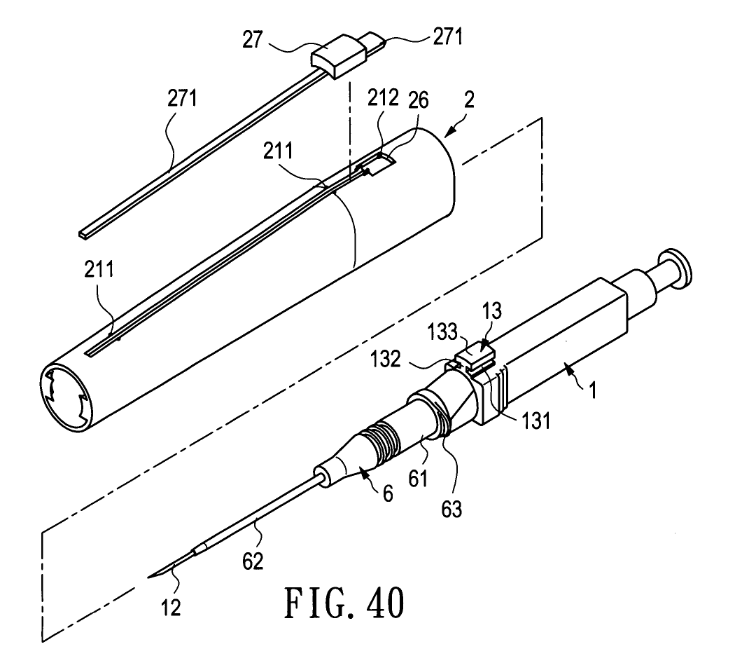

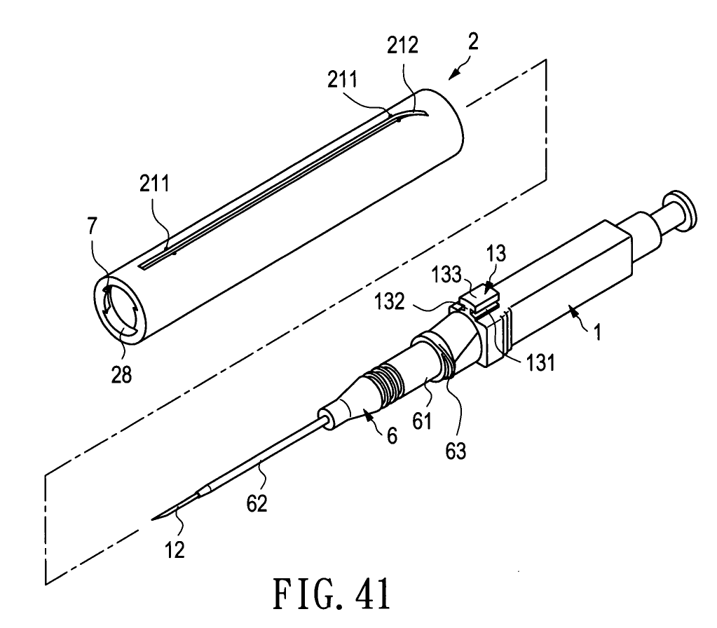

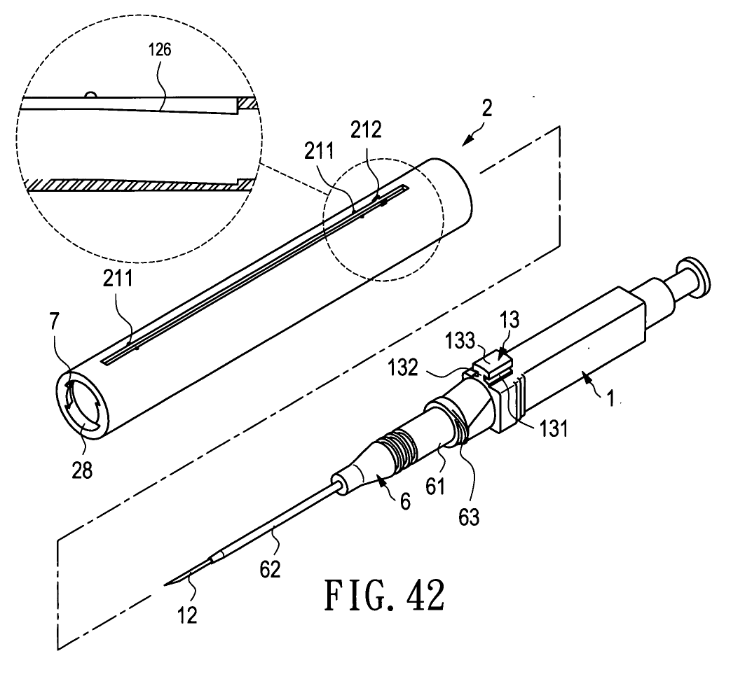

Referring to FIGS. 26, 27, 28, 29, 30, 31, 32, 33, 34, 35, 36, 37, 38, 39, 40, 41, 42 and 43, in the present single-use safety injector structure with the catheter mentioned above, the front-end open portion of the protective cover 2 is provided with a hooking portion 7. Furthermore, the structures of the protective cover 2 and the gliding block 13 are similar to those shown in FIGS. 2, 3, 5, 6, 7, 8, 9, 10, 11, 12, 13, 16, 17, 19, 20, 21, 22 and 23.

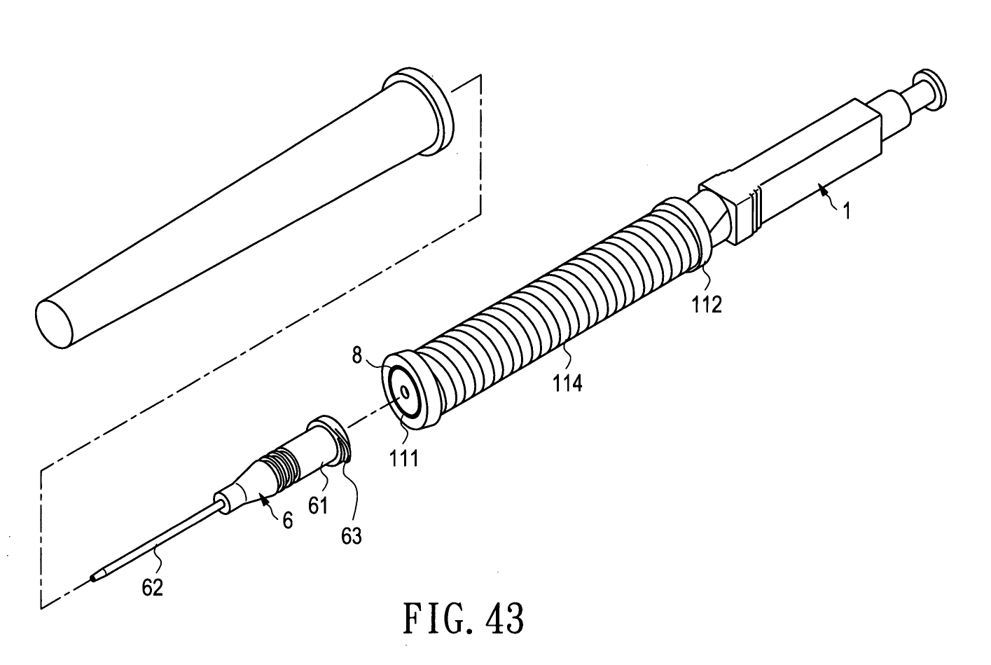

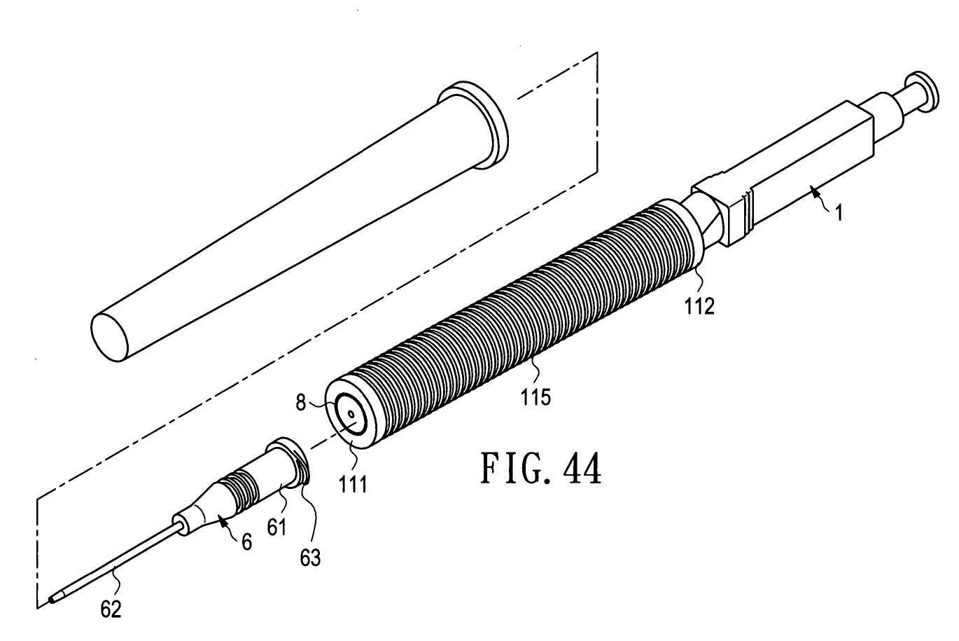

Referring to FIGS. 43 and 44, in the single-use safety injector structure provided by the invention, the ring sheet 114 can be replaced by a retractable tube 115. The axel center of the retractable tube 115 has a bias angle in respect with the axel center of the injection needle 12. As such, when the front protective cover 111 is pulled forwardly, a biasing condition occurs. Furthermore, the front edge of the front protective cover 111 is provided with several connection elements 8, e.g. buckle-hooks or viscose elements, so as to be hooked or bonded with the ring stopping portion 63 of the catheter 6 before use.

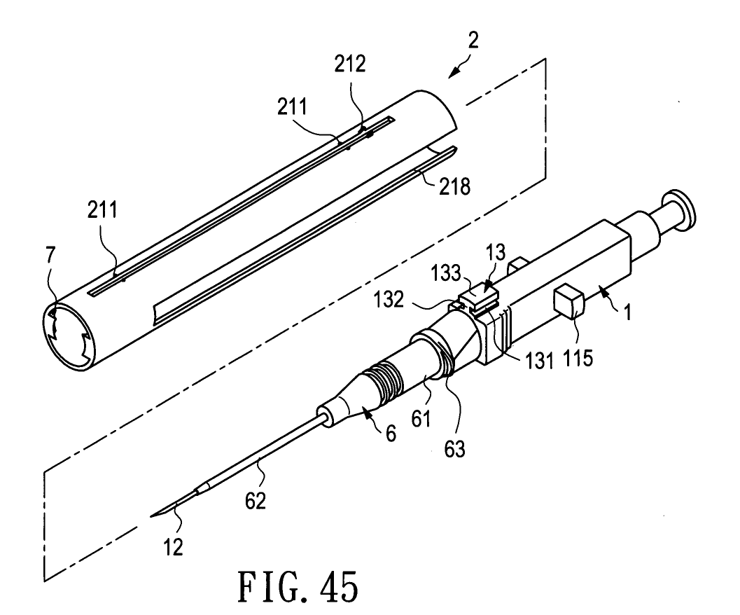

Referring to FIG. 45, in the single-use safety injector structure provided by the invention, the outer periphery of the syringe needle 1 can be convexly provided with several symmetrical handheld portions 115. The protective cover 2 is provided with a gliding slot 218. The handheld portion 115 can penetrate through the gliding slot 218. As such, when the medical practitioner uses the single-use safety injector structure of the invention, the handheld portion 115 can be grasped to avoid uneasy operating conditions resulting from encapsulation of the syringe needle 1 with the protective cover 2. Alternatively, the outer periphery of the syringe needle 1 can be provided with no handheld portion 115, and the protective cover 2 is provided with no gliding slot 218. Alternatively, another gliding block 13 is provided on the opposed side of the gliding block 13 disposed on the outer periphery of the syringe needle 1, and a gliding track is provided at a position corresponding to the gliding block 13. As such, when the medical practitioner uses the single-use safety injector structure of the invention, the upper stopping portions 133 of the two gliding blocks 13 can be grasped to avoid uneasy operating conditions resulting from encapsulation of the syringe needle 1 with the protective cover 2.

Many changes and modifications in the above described embodiment of the invention can, of course, be carried out without departing from the scope thereof. Accordingly, to promote the progress in science and the useful arts, the invention is disclosed and is intended to be limited only by the scope of the appended claims.

1. A single-use safety injector structure comprises:
a syringe needle, having an injection needle inserted into an insertion portion thereof, wherein the outer periphery of said insertion portion is provided with a gliding block, and said gliding block includes a base, a guiding rod and an upper stopping portion;
a protective cover, provided with a gliding track on the outer periphery thereof, wherein one or both sides in the vicinity of the front end and rear end of said gliding track are provided with salients, and said gliding track is provided with one or more non-return stopping blocks behind and in the vicinity of said rear-end salient;
a syringe for inserting the syringe needle via the front end thereof;
wherein said syringe needle is connected to the front end of said syringe via said insertion portion, said protective cover is sleeved around the outer periphery of said syringe needle, by means of said guiding rod, said syringe needle penetrates through said gliding track of said protective cover, by means of said base, said syringe needle is sustained against the inner wall of said protective cover, and by means of said upper stopping portion, said syringe needle is sustained against the external wall of said protective cover.

2. The single-use safety injector structure according to claim 1, wherein said salient and said non-return stopping blocks arranged on the outer periphery of said protective cover is provided on the inner wall of said protective cover or said gliding track.

3. The single-use safety injector structure according to claim 1, wherein the outer periphery of said protective cover is provided with no salient and no non-return stopping block, said gliding track is concavely provided with an opening at the terminal thereof, one or more salients are convexly provided between said gliding track and said opening, and said upper stopping portion of said gliding block on the terminal and lower portion thereof is provided with a raised block to be accommodated within said opening.

4. The single-use safety injector structure according to claim 1, wherein said gliding track of said protective cover in the vicinity of the front end thereof is provided with no salient, and the front end of said gliding track forms a tapered clip portion.

5. The single-use safety injector structure according to claim 1, wherein said protective cover remains the front portion for encapsulating the tip of said injection needle, but the rear portion thereof can be replaced with a sheet or semi-circular body so as to avoid sheltering graduation on the injector.

6. The single-use safety injector structure according to claim 1, wherein said gliding track in the vicinity of the rear end thereof is provided with no salient but directly connected to said upper stopping portion and said protective cover via a connection member, and said connection member is replaced by a viscose or other adhesive element.

7. The single-use safety injector structure according to claim 1, wherein said protective cover is also provided with a whirl slot behind one side of the rear-end non-return stopping block, and several non-return stopping blocks are provided on the connection portion between said whirl slot and said gliding track.

8. The single-use safety injector structure according to claim 7, wherein said non-return stopping block in the vicinity of the front end thereof is also provided with no salient, and one side on the front end of said gliding track is provided with a whirl slot.

9. The single-use safety injector structure according to claim 1, wherein said protective cover is also provided with no non-return stopping block on the rear end of said gliding track, a raised block is extended from one side at the rear end of said gliding track, a fastening slot is provided on said raised block, and said upper stopping portion is provided with a protruding buckle at a position corresponding to said fastening slot of said raised block.

10. The single-use safety injector structure according to claim 9, wherein the position of said fastening slot on said raised block is exchanged with the position of said protruding buckle on said upper stopping portion.

11. The single-use safety injector structure according to claim 9, wherein said raised block is provided at a position near the rear end of said gliding track, no fastening slot is provided on said raised block, and said protruding buckle provided on said upper stopping portion is replaced by a non-return stopping block.

12. The single-use safety injector structure according to claim 11, wherein said non-return stopping block of said upper stopping portion is provided on said raised block.

13. The single-use safety injector structure according to claim 1, wherein no non-return stopping block is provided on the rear end of said gliding track, and the terminal and lower portion of said upper stopping portion extends downwardly a fastening hook to be hooked onto the rear-end inner wall of said protective cover.

14. The single-use safety injector structure according to claim 1, wherein one or both sides of said upper stopping portion extend downwardly a penetrating hook, one or both sides of said gliding track of said protective cover in the vicinity of the front end and the rear end thereof are provided with several recesses, and one or both sides at the terminal of said gliding track of said protective cover are provided with a penetrating hook.

15. The single-use safety injector structure according to claim 1, wherein said gliding track in the vicinity of the front end thereof is provided with no salient, said protective cover is provided with an indenting buckle on the front end of said gliding track, and the front end and lower edge of said upper stopping portion is provided with a protruding buckle, wherein the position of said protruding buckle of said upper stopping portion can be exchanged with the position of said indenting buckle of said protective cover.

16. The single-use safety injector structure according to claim 1, wherein said gliding track in the vicinity of the front end thereof is provided with no salient, the front end and lower edge of said upper stopping portion is provided with a protruding portion, and each of the front and rear ends of said protruding portion comprises an inclined plane.

17. The single-use safety injector structure according to claim 1, wherein said protective cover remains the front portion for encapsulating the tip of said injection needle, but the rear portion thereof is replaced with a sheet body, wherein said syringe is provided with an indenting portion at a position corresponding to said sheet body on the rear portion of said protective cover.

18. The single-use safety injector structure according to claim 1, wherein said protective cover remains the front portion for encapsulating the tip of said injection needle, but the rear portion thereof is replaced with a sheet body, said salients and said non-return stopping block are provided on the inner wall of said gliding track, no upper stopping portion is provided on said gliding block, and an encapsulating portion for accommodating said sheet body on the rear portion of said protective cover is convexly provided on the outer periphery of said syringe.

19. The single-use safety injector structure according to claim 1, wherein said gliding track in the vicinity of the front end thereof is provided with no salient, the front edge of said upper stopping portion is provided with a protruding buckle, the front end of the outer periphery of said protective cover is convexly provided with a raised block, said raised block is provided with an indenting buckle at a position corresponding to said protruding buckle at the front end of the outer periphery of the protective cover, wherein the position of said protruding buckle on said upper stopping portion is exchanged with the position of said indenting buckle on said raised block.

20. The single-use safety injector structure according to claim 19, wherein the rear edge of said upper stopping portion is provided with a protruding buckle, the terminal of the protruding buckle is provided with a plugging portion, the terminal of said protective cover is provided with a raised block having an indenting buckle, and the inner side of said indenting buckle is provided with a buckling portion.

21. The single-use safety injector structure according to claim 1, wherein said insertion portion is provided with no gliding block, said protective cover is consisted of a front protective cover and a rear protective cover, the periphery of the open side of said front protective cover is provided with a ring stopping portion, said front protective cover is connected to said rear protective cover via a ring-shaped sheet, and the axel center of said ring-shaped sheet has a bias angle in respect with the axel center of said front protective cover and said rear protective cover.

22. The single-use safety injector structure according to claim 21, wherein the axel center of said ring-shaped sheet is coaxial with the axel centers of said front protective cover and said rear protective cover, and said ring-shaped sheet is connected to said front protective cover via a single-point or single-side connection manner.

23. The single-use safety injector structure according to claim 1, wherein said gliding block is separated from said insertion portion, said insertion portion is concavely disposed with a ring-shape recess, a fastening hole is provided in the center of said base of said gliding block, and said fastening hole is disposed around the outer periphery of said ring-shape recess.

24. The single-use safety injector structure according to claim 1, wherein the rear end of said non-return stopping block of said protective cover is provided with an accommodating portion, the inner periphery of said accommodating portion is provided with several non-return stopping blocks, and said protective cover is tapered from the rear-end salient of said gliding track to the front end thereof.

25. The single-use safety injector structure according to claim 24, wherein said protective cover is not tapered from the rear-end salient of said gliding track to the front end thereof, but said protective cover is raised from the rear-end salient of said gliding track.

26. The single-use safety injector structure according to claim 24, wherein no rear-end salient is provided on said gliding track, but a cap sheath is covered on said upper stopping portion, and each of the front and the rear ends of said cap sheath extends a cap body to be placed into said gliding track and said accommodating portion.

27. The single-use safety injector structure according to claim 1, wherein the rear end of said gliding track is provided with no non-return stopping block, a bias oblique angle 27 is formed in the inner wall at the rear end of said gliding track or the rear end of said protective cover 2, and the front end of said protective cover is provided with a ring stopping portion for said injection needle to be pierced therethrough.

28. A single-use safety injector structure comprises:
a syringe needle, having an injection needle inserted into the front end thereof, wherein the outer periphery of said syringe needle is provided with a gliding block, and said gliding block includes a base, a guiding rod and an upper stopping portion;
a catheter, comprising a sheathing tube provided with a flexible tube extended from the front end thereof and a ring stopping portion at the rear periphery thereof; and
a protective cover, provided with a gliding track on the outer periphery thereof, wherein one or both sides in the vicinity of the front end and rear end of said gliding track are provided with salients, said gliding track is provided with one or more non-return stopping blocks behind and in the vicinity of said rear-end salient, and the inner periphery of the front-end open portion of said protective cover is provided with several hooking portions,
wherein said catheter is connected to the front end of said syringe needle via said sheathing tube, the outer portion of said syringe needle is covered by said flexible tube, and said protective cover is sleeved around the outer periphery of said syringe needle, wherein by means of said guiding rod, said syringe needle penetrates through said gliding track of said protective cover, by means of said base, said syringe needle is sustained against the inner wall of said protective cover, and by means of said upper stopping portion, said syringe needle is sustained against the external wall of said protective cover.

29. The single-use safety injector structure according to claim 28, wherein the front edge of said protective cover is sustained against the rear side of said sheathing tube, said injection needle is covered by a conventional syringe cover, and the front edge of said protective cover is provided with several connection elements to be hooked or bonded with said ring stopping portion of said catheter.

30. The single-use safety injector structure according to claim 28, wherein said protective cover remains the front portion for encapsulating the tip of said injection needle, but the rear portion thereof is replaced with a sheet body.

31. The single-use safety injector structure according to claim 28, wherein said salient and said non-return stopping blocks arranged on the outer periphery of said protective cover is provided on the inner wall of said protective cover.

32. The single-use safety injector structure according to claim 28, wherein the outer periphery of said protective cover is provided with no salient and no non-return stopping block, said gliding track is concavely provided with an opening at the terminal thereof, and said upper stopping portion of said gliding block on the terminal and lower portion thereof is provided with a raised block to be accommodated within said opening.

33. The single-use safety injector structure according to claim 28, wherein said gliding track of said protective cover in the vicinity of the front end thereof is provided with no salient, and the front end of said gliding track forms a tapered clip portion.

34. The single-use safety injector structure according to claim 28, wherein said gliding track in the vicinity of the rear end thereof is provided with no salient but directly connected to said upper stopping portion and said protective cover via a connection member, and said connection member is replaced by a viscose or other adhesive element.

35. The single-use safety injector structure according to claim 28, wherein said protective cover is also provided with a whirl slot behind one side of the rear-end non-return stopping block, and several non-return stopping blocks are provided on the connection portion between said whirl slot and said gliding track.

36. The single-use safety injector structure according to claim 35, wherein said non-return stopping block in the vicinity of the front end thereof is also provided with no salient, and one side on the front end of said gliding track is provided with a whirl slot.

37. The single-use safety injector structure according to claim 28, wherein said protective cover is also provided with no non-return stopping block on the rear end of said gliding track, a raised block is extended from one side at the rear end of said gliding track, a fastening slot is provided on said raised block, and said upper stopping portion is provided with a protruding buckle at a position corresponding to said fastening slot of said raised block.

38. The single-use safety injector structure according to claim 37, wherein the position of said fastening slot on said raised block is exchanged with the position of said protruding buckle on said upper stopping portion.

39. The single-use safety injector structure according to claim 37, wherein said raised block is provided at a position near the rear end of said gliding track, no fastening slot is provided on said raised block, and said protruding buckle provided on said upper stopping portion is replaced by a non-return stopping block.

40. The single-use safety injector structure according to claim 37, wherein said upper stopping portion is provided with no protruding buckle, said raised block is provided at a position near the rear end of said gliding track, no non-return stopping block is provided on the rear end of said gliding track, and said fastening slot on said raised block is replaced by a non-return stopping block.

41. The single-use safety injector structure according to claim 28, wherein no non-return stopping block is provided on the rear end of said gliding track, and the terminal and lower portion of said upper stopping portion extends downwardly a fastening hook to be hooked onto the rear-end inner wall of said protective cover.

42. The single-use safety injector structure according to claim 28, wherein one or both sides of said upper stopping portion extend downwardly a penetrating hook, one or both sides of said gliding track of said protective cover in the vicinity of the front end and the rear end thereof are provided with several recesses, and one or both sides at the terminal of said gliding track of said protective cover are provided with a penetrating hook.

43. The single-use safety injector structure according to claim 28, wherein said gliding track in the vicinity of the front end thereof is provided with no salient, said protective cover is provided with an indenting buckle on the front end of said gliding track, and the front end and lower edge of said upper stopping portion is provided with a protruding buckle.

44. The single-use safety injector structure according to claim 28, wherein said gliding track in the vicinity of the front end thereof is provided with no salient, the front end and lower edge of said upper stopping portion is provided with a protruding portion, and each of the front and rear ends of said protruding portion comprises an inclined plane.

45. The single-use safety injector structure according to claim 28, wherein the front edge of said upper stopping portion is provided with a protruding buckle, the front end of the outer periphery of said protective cover is convexly provided with a raised block, said raised block is provided with an indenting buckle at a position corresponding to said protruding buckle at the front end of the outer periphery of the protective cover, wherein the position of said protruding buckle on said upper stopping portion is exchanged with the position of said indenting buckle on said raised block.

46. The single-use safety injector structure according to claim 45, wherein the rear edge of said upper stopping portion is provided with a protruding buckle, the terminal of the protruding buckle is provided with a plugging portion, the terminal of said protective cover is provided with a raised block having an indenting buckle, and the inner side of said indenting buckle is provided with a buckling portion.

47. The single-use safety injector structure according to claim 28, wherein the rear end of said non-return stopping block of said protective cover is provided with an accommodating portion, the inner periphery of said accommodating portion is provided with several non-return stopping blocks, and said protective cover is tapered from the rear-end salient of said gliding track to the front end thereof.

48. The single-use safety injector structure according to claim 47, wherein said protective cover is not tapered from the rear-end salient of said gliding track to the front end thereof, but said protective cover is raised from the rear-end salient of said gliding track.

49. The single-use safety injector structure according to claim 47, wherein no rear-end salient is provided on said gliding track, but a cap sheath is covered on said upper stopping portion, and each of the front and the rear ends of said cap sheath extends a cap body to be placed into said gliding track and said accommodating portion.

51. The single-use safety injector structure according to claim 28, wherein said syringe needle is provided with no gliding block, said protective cover is consisted of a front protective cover and a rear protective cover, the periphery of the open side of said front protective cover is provided with a ring stopping portion, said front protective cover is connected to said rear protective cover via a ring-shaped sheet, the axel center of said ring-shaped sheet has a bias angle in respect with the axel center of said front protective cover and said rear protective cover, and the front edge of said front protective cover is provided with several connection elements to be hooked or bonded with said ring stopping portion of said catheter.

52. The single-use safety injector structure according to claim 51, wherein the axel center of said ring-shaped sheet is coaxial with the axel centers of said front protective cover and said rear protective cover, and said ring-shaped sheet is connected to said front protective cover via a single-point or single-side connection manner.

53. The single-use safety injector structure according to claim 51, wherein said ring sheet is replaced by a retractable tube, the axel center of said retractable tube has a bias angle in respect with the axel center of said injection needle.

54. The single-use safety injector structure according to claim 28, wherein the outer periphery of said syringe needle is convexly provided with several symmetrical handheld portions, and said protective cover is provided with a gliding slot for said handheld portion to penetrate therethrough.

55. The single-use safety injector structure according to claim 28, wherein another gliding block is provided on the opposed side of said gliding block disposed on the outer periphery of said syringe needle, and a gliding track is provided at a position corresponding to said gliding block.
